(19) United States
(12) Patent Application Publication
(10) Pub. No.: US 2026/0183086 A1
(43) Pub. Date: Jul. 2, 2026

(54) ORAL HEALTH PROGNOSIS SYSTEM

(71) Applicant: Align Technology, Inc., San Jose, CA (US)

(72) Inventors: Moti Ben Dov, Tel Mond (IL); Ran Katz, Hod Hasharon (IL)

(21) Appl. No.: 19/431,307

(22) Filed: Dec. 23, 2025

Related U.S. Application Data
(60) Provisional application No. 63/741,292, filed on Jan. 2, 2025.

(51) Int. Cl. A61C 7/00; A61B 1/24; A61C 7/08; G16H 50/20

(52) U.S. Cl. CPC A61C 7/002; A61B 1/24; A61C 7/08; G16H 50/20

(57) ABSTRACT

A system comprising an intraoral scanner configured to generate scan data of a dental arch of a patient, and a computing device operatively coupled to the intraoral scanner, the computing device to provide oral health prognosis. In some cases, the computing device can receive the scan data. The computing device can provide the scan data as input to an artificial intelligence (AI) model trained to output a value indicating a predicted dental condition. The computing device can provide, to a user device, an indication of the predicted dental condition based on the output provided by the AI model.

### RELATED APPLICATION

The present application claims priority to U.S. Provisional Patent Application No. 63/741,292, filed on Jan. 2, 2025, which is herein incorporated by reference in their entirety.

### TECHNICAL FIELD

The instant specification generally relates to systems and methods for providing oral health prognosis.

### BACKGROUND

Oral health can involve monitoring and maintaining the integrity of oral structures, which may be subject to various progressive conditions throughout a patient's lifetime. Progressive dental conditions can refer to changes in oral structures that occur over time. These conditions may affect various components of the oral cavity, including teeth, gingiva, and supporting tissues. Progressive dental conditions can include tooth wear (such as attrition, erosion, abrasion, and abfraction), gingival recession, gingival inflammation, structural changes to tooth surfaces, and other conditions that manifest as changes in dental arch geometry or appearance. Such conditions can be caused by various mechanical, chemical, and/or biological factors. For example, tooth wear can be caused by tooth-to-tooth contact, such as teeth grinding or bruxism, chemical acids that can soften and wear away the enamel of the tooth, and/or poor dental hygiene, for example. Gingival recession can result from periodontal disease, aggressive brushing, or anatomical factors. Progressive dental conditions can lead to various clinical manifestations, including increased tooth sensitivity, changes in the shape of the tooth, tooth discoloration, and structural alterations.

Progressive dental conditions can be assessed through patient history, clinical examination, and/or diagnostic tools, such as x-rays, intraoral scans and digital imaging. A dental professional (e.g., a dentist) can evaluate current conditions by examining wear patterns, tissue changes, and other clinical indictors. Assessment of progressives dental conditions may involve identifying contributing risk factors, and/or monitoring progression over time. Evaluation of such conditions can involve comparing current clinical presentations with previous records obtained during earlier patient visits.

### SUMMARY

The below summary is a simplified summary of the disclosure in order to provide a basic understanding of some aspects of the disclosure. This summary is not an extensive overview of the disclosure. It is intended neither to identify key or critical elements of the disclosure, nor delineate any scope of the particular embodiments of the disclosure or any scope of the claims. Its sole purpose is to present some concepts of the disclosure in a simplified form as a prelude to the more detailed description that is presented later.

In a first implementation, a system comprises an intraoral scanner configured to generate scan data of a dental arch of a patient, and a computing device operatively coupled to the intraoral scanner, the computing device configured to receive the scan data, provide the scan data as input to an artificial intelligence (AI) model trained to output a value indicating a predicted dental condition, and provide, to a user device, an indication of the predicted dental condition based on the output provided by the AI model.

A second implementation may further extend the first implementation. In the second implementation, the predicted dental condition comprises at least one of: tooth wear, gum recession, gingival inflammation, chipped tooth, bruxism-related damage, gastroesophageal reflux disease (GERD) effects, or a missing attachment.

A third implementation may further extend the first through second implementations. In the third implementation, the scan data comprises color data, and the predicted dental condition comprises at least one of tooth staining, loss of vitality, poor oral care indicator, fluorosis, tooth decay, gingivitis, or a soft tissue lesion.

A fourth implementation may further extend the first through third implementations. In the fourth implementation, the indication corresponds to the output from the AI model.

A fifth implementation may further extend the first through fourth implementations. In the fifth implementation, the indication is determined based on processing the output of the AI model using a function.

A sixth implementation may further extend the first through fifth implementations. In the sixth implementation, the computing device is further configured to receive, from the AI model, the value indicating the predicted dental condition, and determine, based on the value, the indication of the predicted dental condition.

A seventh implementation may further extend the first through sixth implementations. In the seventh implementation, the scan data of the dental arch of the patient comprises scan data collected from a first scan performed at a first point in time, and does not comprise scan data collected from a second scan performed at a second point in time.

An eighth implementation may further extend the first through seventh implementations. In the eighth implementation, the AI model is trained using a training dataset that has been automatically labeled with labels indicating a future dental condition based on a comparison of first scan data collected during a first scan performed at a first point in time to second scan data collected during a second scan performed at a second point in time, wherein the comparison indicates progression of a dental condition.

A ninth implementation may further extend the first through eighth implementations. In the ninth implementation, the comparison is used to identify an inconsistency between the first scan data and the second scan data that satisfies a criterion.

A tenth implementation may further extend the first through ninth implementations. In the tenth implementation, the inconsistency satisfies the criterion if a list of predetermined causes comprises a cause of the inconsistency.

An eleventh implementation may further extend the first through tenth implementations. In the eleventh implementation, the inconsistency satisfies the criterion if a size of the inconsistency exceeds a threshold size.

A twelfth implementation may further extend the first through eleventh implementations. In the twelfth implementation, the inconsistency corresponds to at least one of tooth wear, gum recession, chipped tooth, bruxism, gastroesophageal reflux disease (GERD), gingival inflammation, or a missing attachment.

A thirteenth implementation may further extend the first through twelfth implementations. In the thirteenth implementation, the first scan data and the second scan data comprise color data, and the inconsistency corresponds to at least one of tooth staining, loss of vitality, poor oral care indicator, fluorosis, tooth decay, gingivitis, or a soft tissue lesion.

A fourteenth implementation may further extend the first through thirteenth implementations. In the fourteenth implementation, the indication of the predicted dental condition comprises an estimated amount of a progress of the predicted dental condition.

A fifteenth implementation may further extend the first through fourteenth implementations. In the fifteenth implementation, the indication of the predicted dental condition comprises the estimated amount of the progress of the predicted dental condition for a particular time period.

A sixteenth implementation may further extend the first through fifteenth implementations. In the sixteenth implementation, the scan data is segmented into at least one of individual teeth or gingival regions before being provided to the AI model, and the indication of the predicted dental condition corresponds to at least one of a particular tooth or a particular gingival region.

A seventeenth implementation may further extend the first through sixteenth implementations. In the seventeenth implementation, the indication provided to the user device corresponds to a first value of a plurality of values provided by the AI model, wherein the first value corresponds to a highest estimated amount of a progress of the predicted dental condition.

An eighteenth implementation may further extend the first through seventeenth implementations. In the eighteenth implementation, a heat map is provided for display on the user device, wherein the heat map reflects an estimated amount of a progress of the predicted dental condition corresponding to the value indicating the predicted dental condition.

A nineteenth implementation may further extend the first through eighteenth implementations. In the nineteenth implementation, the estimated amount of the progress of the predicted dental condition corresponds to an aggregation of a plurality of estimated amounts of progress of the predicted dental condition, wherein the plurality of estimated amounts of progress of the predicted dental condition correspond to at least a subset of a plurality of values indicating predicted dental condition provided by the AI model, and wherein the subset corresponds to a particular tooth of the dental arch of the patient.

A twentieth implementation may further extend the first through nineteenth implementations. In the twentieth implementation, the AI model is a generative AI model that is trained to generate a three-dimensional mesh to display the value indicating the predicted dental condition on at least one of a corresponding tooth or a corresponding gingival region.

A twenty-first implementation may further extend the first through twentieth implementations. In the twenty-first implementation, the scan data comprises at least one of: one or more three-dimensional mesh, one or more two-dimensional scan, or one or more occlusal maps.

A twenty-second implementation may further extend the first through twenty-first implementations. In the twenty-second implementation, the scan data comprises data from multiple bite positions of the dental arch of the patient.

A twenty-third implementation may further extend the first through twenty-second implementations. In the twenty-third implementation, the scan data comprises information on opposing teeth.

A twenty-fourth implementation may further extend the first through twenty-third implementations. In the twenty-fourth implementation, the computing device is further configured to provide, as further input to the AI model, a time frame for the predicted dental condition, wherein the output from the AI model corresponds to the time frame.

A twenty-fifth implementation may further extend the first through twenty-fourth implementations. In the twenty-fifth implementation, the time frame is received from the user device.

A twenty-sixth implementation may further extend the first through twenty-fifth implementations. In the twenty-sixth implementation, the indication of the predicted dental condition comprises a range of values corresponding to a severity range of the predicted dental condition.

A twenty-seventh implementation may further extend the first through twenty-sixth implementations. In the twenty-seventh implementation, the computing device is further configured to provide, for display on the user device, a first value of the range of values, wherein the first value corresponds to the an indication of the severity range, wherein the indication is received from the user device, and wherein the first value of the range of values reflects the severity range of the predicted dental condition.

A twenty-eighth implementation may further extend the first through twenty-seventh implementations. In the twenty-eighth implementation, a prognosis factor corresponds to an inconsistency between updated scan data of the dental arch and the indication of the predicted dental condition, wherein the inconsistency is based on a comparison of the updated scan data of the dental arch to the indication of the predicted dental condition, and wherein a timing of the updated scan data corresponds to the indication of the predicted dental condition based on output provided by the AI model.

A twenty-ninth implementation may further extend the first through twenty-eighth implementations. In the twenty-ninth implementation, the computing device is further configured to provide, as further input to the AI model, the updated scan data, and provide, to the user device, an updated indication of the predicted dental condition based on updated output provided by the AI model modified by the prognosis factor corresponding to the inconsistency between the updated scan data and the indication of the predicted dental condition.

A thirtieth implementation may further extend the first through twenty-ninth implementations. In the thirtieth implementation, the system further comprises the user device, configured to output the indication of the predicted dental condition.

In a thirty-first implementation, a method comprises receiving scan data of a dental arch of a patient, providing the scan data as input to an artificial intelligence (AI) model trained to output a value indicating a predicted dental condition, and providing, to a user device, an indication of the predicted dental condition based on the output provided by the AI model.

A thirty-second implementation may further extend the thirty-first implementation. In the thirty-second implementation, the predicted dental condition comprises at least one of: tooth wear, gum recession, gingival inflammation, chipped tooth, bruxism-related damage, gastroesophageal reflux disease (GERD) effects, or a missing attachment.

A thirty-third implementation may further extend the thirty-first through thirty-second implementations. In the thirty-third implementation, the scan data comprises color data, and the predicted dental condition comprises at least one of tooth staining, loss of vitality, poor oral care indicator, fluorosis, tooth decay, gingivitis, or a soft tissue lesion.

A thirty-fourth implementation may further extend the thirty-first through thirty-third implementations. In the thirty-fourth implementation, the indication corresponds to the output from the AI model.

A thirty-fifth implementation may further extend the thirty-first through thirty-fourth implementations. In the thirty-fifth implementation, the indication is determined based on processing the output of the AI model using a function.

A thirty-sixth implementation may further extend the thirty-first through thirty-fifth implementations. In the thirty-sixth implementation, the method further comprises receiving, from the AI model, the value indicating the predicted dental condition, and determining, based on the value, the indication of the predicted dental condition.

A thirty-seventh implementation may further extend the thirty-first through thirty-sixth implementations. In the thirty-seventh implementation, the scan data of the dental arch of the patient comprises scan data collected from a first scan performed at a first point in time, and does not comprise scan data collected from a second scan performed at a second point in time.

A thirty-eighth implementation may further extend the thirty-first through thirty-seventh implementations. In the thirty-eighth implementation, the AI model is trained using a training dataset that has been automatically labeled with labels indicating a future dental condition based on a comparison of first scan data collected during a first scan performed at a first point in time to second scan data collected during a second scan performed at a second point in time, wherein the comparison indicates progression of a dental condition.

A thirty-ninth implementation may further extend the thirty-first through thirty-eighth implementations. In the thirty-ninth implementation, the comparison is used to identify an inconsistency between the first scan data and the second scan data that satisfies a criterion.

A fortieth implementation may further extend the thirty-first through thirty-ninth implementations. In the fortieth implementation, the inconsistency satisfies the criterion if a list of predetermined causes comprises a cause of the inconsistency.

A forty-first implementation may further extend the thirty-first through fortieth implementations. In the forty-first implementation, the inconsistency satisfies the criterion if a size of the inconsistency exceeds a threshold size.

A forty-second implementation may further extend the thirty-first through forty-first implementations. In the forty-second implementation, the inconsistency corresponds to at least one of tooth wear, gum recession, chipped tooth, bruxism, gastroesophageal reflux disease (GERD), gingival inflammation, or a missing attachment.

A forty-third implementation may further extend the thirty-first through forty-second implementations. In the forty-third implementation, the first scan data and the second scan data comprise color data, and the inconsistency corresponds to at least one of tooth staining, loss of vitality, poor oral care indicator, fluorosis, tooth decay, gingivitis, or a soft tissue lesion.

A forty-fourth implementation may further extend the thirty-first through forty-third implementations. In the forty-fourth implementation, the indication of the predicted dental condition comprises an estimated amount of a progress of the predicted dental condition.

A forty-fifth implementation may further extend the thirty-first through forty-fourth implementations. In the forty-fifth implementation, the indication of the predicted dental condition comprises the estimated amount of the progress of the predicted dental condition for a particular time period.

A forty-sixth implementation may further extend the thirty-first through forty-fifth implementations. In the forty-sixth implementation, the scan data is segmented into at least one of individual teeth or gingival regions before being provided to the AI model, and the indication of the predicted dental condition corresponds to at least one of a particular tooth or a particular gingival region.

A forty-seventh implementation may further extend the thirty-first through forty-sixth implementations. In the forty-seventh implementation, the indication provided to the user device corresponds to a first value of a plurality of values provided by the AI model, wherein the first value corresponds to a highest estimated amount of a progress of the predicted dental condition.

A forty-eighth implementation may further extend the thirty-first through forty-seventh implementations. In the forty-eighth implementation, a heat map is provided for display on the user device, wherein the heat map reflects an estimated amount of a progress of the predicted dental condition corresponding to the value indicating the predicted dental condition.

A forty-ninth implementation may further extend the thirty-first through forty-eighth implementations. In the forty-ninth implementation, the estimated amount of the progress of the predicted dental condition corresponds to an aggregation of a plurality of estimated amounts of progress of the predicted dental condition, wherein the plurality of estimated amounts of progress of the predicted dental condition correspond to at least a subset of a plurality of values indicating predicted dental condition provided by the AI model, and wherein the subset corresponds to a particular tooth of the dental arch of the patient.

A fiftieth implementation may further extend the thirty-first through forty-ninth implementations. In the fiftieth implementation, the AI model is a generative AI model that is trained to generate a three-dimensional mesh to display the value indicating the predicted dental condition on at least one of a corresponding tooth or a corresponding gingival region.

A fifty-first implementation may further extend the thirty-first through fiftieth implementations. In the fifty-first implementation, the scan data comprises at least one of: one or more three-dimensional mesh, one or more two-dimensional scan, or one or more occlusal maps.

A fifty-second implementation may further extend the thirty-first through fifty-first implementations. In the fifty-second implementation, the scan data comprises data from multiple bite positions of the dental arch of the patient.

A fifty-third implementation may further extend the thirty-first through fifty-second implementations. In the fifty-third implementation, the scan data comprises information on opposing teeth.

A fifty-fourth implementation may further extend the thirty-first through fifty-third implementations. In the fifty-fourth implementation, the method further comprises providing, as further input to the AI model, a time frame for the predicted dental condition, wherein the output from the AI model corresponds to the time frame.

A fifty-fifth implementation may further extend the thirty-first through fifty-fourth implementations. In the fifty-fifth implementation, the time frame is received from the user device.

A fifty-sixth implementation may further extend the thirty-first through fifty-fifth implementations. In the fifty-sixth implementation, the indication of the predicted dental condition comprises a range of values corresponding to a severity range of the predicted dental condition.

A fifty-seventh implementation may further extend the thirty-first through fifty-sixth implementations. In the fifty-seventh implementation, the method further comprises providing, for display on the user device, a first value of the range of values, wherein the first value corresponds to the an indication of the severity range, wherein the indication is received from the user device, and wherein the first value of the range of values reflects the severity range of the predicted dental condition.

A fifty-eighth implementation may further extend the thirty-first through fifty-seventh implementations. In the fifty-eighth implementation, a prognosis factor corresponds to an inconsistency between updated scan data of the dental arch and the indication of the predicted dental condition, wherein the inconsistency is based on a comparison of the updated scan data of the dental arch to the indication of the predicted dental condition, and wherein a timing of the updated scan data corresponds to the indication of the predicted dental condition based on output provided by the AI model.

A fifty-ninth implementation may further extend the thirty-first through fifty-eighth implementations. In the fifty-ninth implementation, the method further comprises providing, as further input to the AI model, the updated scan data, and providing, to the user device, an updated indication of the predicted dental condition based on updated output provided by the AI model modified by the prognosis factor corresponding to the inconsistency between the updated scan data and the indication of the predicted dental condition.

A sixtieth implementation may further extend the thirty-first through fifty-ninth implementations. In the sixtieth implementation, the method is performed by a computing device operatively coupled to an intraoral scanner configured to generate the scan data of the dental arch of the patient.

In a sixty-first implementation, a non-transitory computer-readable storage medium comprises instructions that, when executed by a processing device, cause the processing device to receive scan data of a dental arch of a patient, provide the scan data as input to an artificial intelligence (AI) model trained to output a value indicating a predicted dental condition, and provide, to a user device, an indication of the predicted dental condition based on the output provided by the AI model.

A sixty-second implementation may further extend the sixty-first implementation. In the sixty-second implementation, the predicted dental condition comprises at least one of: tooth wear, gum recession, gingival inflammation, chipped tooth, bruxism-related damage, gastroesophageal reflux disease (GERD) effects, or a missing attachment.

A sixty-third implementation may further extend the sixty-first through sixty-second implementations. In the sixty-third implementation, the scan data comprises color data, and the predicted dental condition comprises at least one of tooth staining, loss of vitality, poor oral care indicator, fluorosis, tooth decay, gingivitis, or a soft tissue lesion.

A sixty-fourth implementation may further extend the sixty-first through sixty-third implementations. In the sixty-fourth implementation, the indication corresponds to the output from the AI model.

A sixty-fifth implementation may further extend the sixty-first through sixty-fourth implementations. In the sixty-fifth implementation, the indication is determined based on processing the Output of the AI model using a function.

A sixty-sixth implementation may further extend the sixty-first through sixty-fifth implementations. In the sixty-sixth implementation, the processing device is further to receive, from the AI model, the value indicating the predicted dental condition, and determine, based on the value, the indication of the predicted dental condition.

A sixty-seventh implementation may further extend the sixty-first through sixty-sixth implementations. In the sixty-seventh implementation, the scan data of the dental arch of the patient comprises scan data collected from a first scan performed at a first point in time, and does not comprise scan data collected from a second scan performed at a second point in time.

A sixty-eighth implementation may further extend the sixty-first through sixty-seventh implementations. In the sixty-eighth implementation, the AI model is trained using a training dataset that has been automatically labeled with labels indicating a future dental condition based on a comparison of first scan data collected during a first scan performed at a first point in time to second scan data collected during a second scan performed at a second point in time, wherein the comparison indicates progression of a dental condition.

A sixty-ninth implementation may further extend the sixty-first through sixty-eighth implementations. In the sixty-ninth implementation, the comparison is used to identify an inconsistency between the first scan data and the second scan data that satisfies a criterion.

A seventieth implementation may further extend the sixty-first through sixty-ninth implementations. In the seventieth implementation, the inconsistency satisfies the criterion if a list of predetermined causes comprises a cause of the inconsistency.

A seventy-first implementation may further extend the sixty-first through seventieth implementations. In the seventy-first implementation, the inconsistency satisfies the criterion if a size of the inconsistency exceeds a threshold size.

A seventy-second implementation may further extend the sixty-first through seventy-first implementations. In the seventy-second implementation, the inconsistency corresponds to at least one of tooth wear, gum recession, chipped tooth, bruxism, gastroesophageal reflux disease (GERD), gingival inflammation, or a missing attachment.

A seventy-third implementation may further extend the sixty-first through seventy-second implementations. In the seventy-third implementation, the first scan data and the second scan data comprise color data, and the inconsistency corresponds to at least one of tooth staining, loss of vitality, poor oral care indicator, fluorosis, tooth decay, gingivitis, or a soft tissue lesion.

A seventy-fourth implementation may further extend the sixty-first through seventy-third implementations. In the seventy-fourth implementation, the indication of the predicted dental condition comprises an estimated amount of a progress of the predicted dental condition.

A seventy-fifth implementation may further extend the sixty-first through seventy-fourth implementations. In the seventy-fifth implementation, the indication of the predicted dental condition comprises the estimated amount of the progress of the predicted dental condition for a particular time period.

A seventy-sixth implementation may further extend the sixty-first through seventy-fifth implementations. In the seventy-sixth implementation, the scan data is segmented into at least one of individual teeth or gingival regions before being provided to the AI model, and the indication of the predicted dental condition corresponds to at least one of a particular tooth or a particular gingival region.

A seventy-seventh implementation may further extend the sixty-first through seventy-sixth implementations. In the seventy-seventh implementation, the indication provided to the user device corresponds to a first value of a plurality of values provided by the AI model, wherein the first value corresponds to a highest estimated amount of a progress of the predicted dental condition.

A seventy-eighth implementation may further extend the sixty-first through seventy-seventh implementations. In the seventy-eighth implementation, a heat map is provided for display on the user device, wherein the heat map reflects an estimated amount of a progress of the predicted dental condition corresponding to the value indicating the predicted dental condition.

A seventy-ninth implementation may further extend the sixty-first through seventy-eighth implementations. In the seventy-ninth implementation, the estimated amount of the progress of the predicted dental condition corresponds to an aggregation of a plurality of estimated amounts of progress of the predicted dental condition, wherein the plurality of estimated amounts of progress of the predicted dental condition correspond to at least a subset of a plurality of values indicating predicted dental condition provided by the AI model, and wherein the subset corresponds to a particular tooth of the dental arch of the patient.

An eightieth implementation may further extend the sixty-first through seventy-ninth implementations. In the eightieth implementation, the AI model is a generative AI model that is trained to generate a three-dimensional mesh to display the value indicating the predicted dental condition on at least one of a corresponding tooth or a corresponding gingival region.

An eighty-first implementation may further extend the sixty-first through eightieth implementations. In the eighty-first implementation, the scan data comprises at least one of: one or more three-dimensional mesh, one or more two-dimensional scan, or one or more occlusal maps.

An eighty-second implementation may further extend the sixty-first through eighty-first implementations. In the eighty-second implementation, the scan data comprises data from multiple bite positions of the dental arch of the patient.

An eighty-third implementation may further extend the sixty-first through eighty-second implementations. In the eighty-third implementation, the scan data comprises information on opposing teeth.

An eighty-fourth implementation may further extend the sixty-first through eighty-third implementations. In the eighty-fourth implementation, the processing device is further to provide, as further input to the AI model, a time frame for the predicted dental condition, wherein the output from the AI model corresponds to the time frame.

An eighty-fifth implementation may further extend the sixty-first through eighty-fourth implementations. In the eighty-fifth implementation, the time frame is received from the user device.

An eighty-sixth implementation may further extend the sixty-first through eighty-fifth implementations. In the eighty-sixth implementation, the indication of the predicted dental condition comprises a range of values corresponding to a severity range of the predicted dental condition.

An eighty-seventh implementation may further extend the sixty-first through eighty-sixth implementations. In the eighty-seventh implementation, the processing device is further to provide, for display on the user device, a first value of the range of values, wherein the first value corresponds to the an indication of the severity range, wherein the indication is received from the user device, and wherein the first value of the range of values reflects the severity range of the predicted dental condition.

An eighty-eighth implementation may further extend the sixty-first through eighty-seventh implementations. In the eighty-eighth implementation, a prognosis factor corresponds to an inconsistency between updated scan data of the dental arch and the indication of the predicted dental condition, wherein the inconsistency is based on a comparison of the updated scan data of the dental arch to the indication of the predicted dental condition, and wherein a timing of the updated scan data corresponds to the indication of the predicted dental condition based on output provided by the AI model.

An eighty-ninth implementation may further extend the sixty-first through eighty-eighth implementations. In the eighty-ninth implementation, the processing device is further to provide, as further input to the AI model, the updated scan data, and provide, to the user device, an updated indication of the predicted dental condition based on updated output provided by the AI model modified by the prognosis factor corresponding to the inconsistency between the updated scan data and the indication of the predicted dental condition.

A ninetieth implementation may further extend the sixty-first through eighty-ninth implementations. In the ninetieth implementation, the processing device is operatively coupled to an intraoral scanner configured to generate the scan data of the dental arch of the patient.

In a ninety-first implementation, a method comprises identifying first scan data and second scan data of a dental arch of a patient, wherein the first scan data predates the second scan data, segmenting the first scan data to identify one or more sections of the dental arch of the patient, segmenting the second scan data to identify the one or more sections of the dental arch of the patient, making a comparison of a first section of the one or more sections in the first scan data to the first section of the one or more sections in the second scan data, identifying, based on the comparison, an inconsistency for the first section between the first scan data and the second scan data, responsive to determining that the inconsistency for the first section satisfies a criterion, adding to the first scan data an indication of a future dental condition corresponding to the first section, and training, using the first scan data comprising the indication of the future dental condition, an artificial intelligence (AI) model to provide an output comprising one or more indications of a predicted dental condition.

A ninety-second implementation may further extend the ninety-first implementation. In the ninety-second implementation, the method further comprises generating a training dataset comprising the first scan data and a plurality of inconsistencies, wherein the plurality of inconsistencies comprise the inconsistency for the first section, and retraining, using the training dataset, the AI model to provide the output comprising the one or more indications of the predicted dental condition.

A ninety-third implementation may further extend the ninety-first through ninety-second implementations. In the ninety-third implementation, determining that the inconsistency for the first section satisfies the criterion comprises identifying a cause of the inconsistency, and determining that the cause corresponds to a list of predetermined causes.

A ninety-fourth implementation may further extend the ninety-first through ninety-third implementations. In the ninety-fourth implementation, determining that the inconsistency for the first section satisfies the criterion comprises identifying a size of the inconsistency, and determining that the size exceeds a threshold size.

A ninety-fifth implementation may further extend the ninety-first through ninety-fourth implementations. In the ninety-fifth implementation, the method further comprises generating a delta map comprising the inconsistency corresponding to each of the one or more sections of the dental arch of the patient.

A ninety-sixth implementation may further extend the ninety-first through ninety-fifth implementations. In the ninety-sixth implementation, the method further comprises normalizing the delta map to a predetermined time period.

A ninety-seventh implementation may further extend the ninety-first through ninety-sixth implementations. In the ninety-seventh implementation, the future dental condition comprises at least one of tooth wear, gum recession, chipped tooth, bruxism-related damage, gastroesophageal reflux disease (GERD) effects, gingival inflammation, or a missing attachment.

A ninety-eighth implementation may further extend the ninety-first through ninety-seventh implementations. In the ninety-eighth implementation, the first scan data and the second scan data comprise color data, and the inconsistency corresponds to at least one of tooth staining, loss of vitality, poor oral care indicator, fluorosis, tooth decay, gingivitis, or a soft tissue lesion.

In a ninety-ninth implementation, a system comprises one or more computing devices each comprising a memory and one or more processing devices, wherein the one or more computing devices are configured to identify first scan data and second scan data of a dental arch of a patient, wherein the first scan data predates the second scan data, segment the first scan data to identify one or more sections of the dental arch of the patient, segment the second scan data to identify the one or more sections of the dental arch of the patient, make a comparison of a first section of the one or more sections in the first scan data to the first section of the one or more sections in the second scan data, identify, based on the comparison, an inconsistency for the first section between the first scan data and the second scan data, responsive to determining that the inconsistency for the first section satisfies a criterion, add to the first scan data an indication of a future dental condition corresponding to the first section, and train, using the first scan data comprising the indication of the future dental condition, an artificial intelligence (AI) model to provide an output comprising one or more indications of the predicted dental condition.

A one hundredth implementation may further extend the ninety-ninth implementation. In the one hundredth implementation, the one or more computing devices are further configured to generate a training dataset comprising the first scan data and a plurality of inconsistencies, wherein the plurality of inconsistencies comprise the inconsistency for the first section, and retrain, using the training dataset, the AI model to provide the output comprising the one or more indications of the predicted dental condition.

A one hundred first implementation may further extend the ninety-ninth through one hundredth implementations. In the one hundred first implementation, to determine that the inconsistency for the first section satisfies the criterion, the one or more computing devices are further configured to identify a cause of the inconsistency, and determine that the cause corresponds to a list of predetermined causes.

A one hundred second implementation may further extend the ninety-ninth through one hundred first implementations. In the one hundred second implementation, to determine that the inconsistency for the first section satisfies the criterion, the one or more computing devices are further configured to identify a size of the inconsistency, and determine that the size exceeds a threshold size.

A one hundred third implementation may further extend the ninety-ninth through one hundred second implementations. In the one hundred third implementation, the one or more computing devices are further configured to generate a delta map comprising the inconsistency corresponding to each of the one or more sections of the dental arch of the patient.

A one hundred fourth implementation may further extend the ninety-ninth through one hundred third implementations. In the one hundred fourth implementation, the one or more computing devices are further configured to normalize the delta map to a predetermined time period.

A one hundred fifth implementation may further extend the ninety-ninth through one hundred fourth implementations. In the one hundred fifth implementation, the future dental condition comprises at least one of tooth wear, gum recession, chipped tooth, bruxism-related damage, gastroesophageal reflux disease (GERD) effects, gingival inflammation, or a missing attachment.

A one hundred sixth implementation may further extend the ninety-ninth through one hundred fifth implementations. In the one hundred sixth implementation, the first scan data and the second scan data comprise color data, and the inconsistency corresponds to at least one of tooth staining, loss of vitality, poor oral care indicator, fluorosis, tooth decay, gingivitis, or a soft tissue lesion.

In a one hundred seventh implementation, a non-transitory computer-readable storage medium comprises instructions that, when executed by a processing device, cause the processing device to identify first scan data and second scan data of a dental arch of a patient, wherein the first scan data predates the second scan data, segment the first scan data to identify one or more sections of the dental arch of the patient, segment the second scan data to identify the one or more sections of the dental arch of the patient, make a comparison of a first section of the one or more sections in the first scan data to the first section of the one or more sections in the second scan data, identify, based on the comparison, an inconsistency for the first section between the first scan data and the second scan data, responsive to determining that the inconsistency for the first section satisfies a criterion, add to the first scan data an indication of a future dental condition corresponding to the first section, and train, using the first scan data comprising the indication of the future dental condition, an artificial intelligence (AI) model to provide an output comprising one or more indications of the predicted dental condition.

A one hundred eighth implementation may further extend the one hundred seventh implementation. In the one hundred eighth implementation, the processing device is further to generate a training dataset comprising the first scan data and a plurality of inconsistencies, wherein the plurality of inconsistencies comprise the inconsistency for the first section, and retrain, using the training dataset, the AI model to provide the output comprising the one or more indications of the predicted dental condition.

A one hundred ninth implementation may further extend the one hundred seventh through one hundred eighth implementations. In the one hundred ninth implementation, to determine that the inconsistency for the first section satisfies the criterion the processing device is further to identify a cause of the inconsistency, and determine that the cause corresponds to a list of predetermined causes.

A one hundred tenth implementation may further extend the one hundred seventh through one hundred ninth implementations. In the one hundred tenth implementation, to determine that the inconsistency for the first section satisfies the criterion the processing device is further to identify a size of the inconsistency, and determine that the size exceeds a threshold size.

A one hundred eleventh implementation may further extend the one hundred seventh through one hundred tenth implementations. In the one hundred eleventh implementation, the processing device is further to generate a delta map comprising the inconsistency corresponding to each of the one or more sections of the dental arch of the patient.

A one hundred twelfth implementation may further extend the one hundred seventh through one hundred eleventh implementations. In the one hundred twelfth implementation, the processing device is further to normalize the delta map to a predetermined time period.

A one hundred thirteenth implementation may further extend the one hundred seventh through one hundred twelfth implementations. In the one hundred thirteenth implementation, the future dental condition comprises at least one of tooth wear, gum recession, chipped tooth, bruxism-related damage, gastroesophageal reflux disease (GERD) effects, gingival inflammation, or a missing attachment.

A one hundred fourteenth implementation may further extend the one hundred seventh through one hundred thirteenth implementations. In the one hundred fourteenth implementation, the first scan data and the second scan data comprise color data, and the inconsistency corresponds to at least one of tooth staining, loss of vitality, poor oral care indicator, fluorosis, tooth decay, gingivitis, or a soft tissue lesion.

In a one hundred fifteenth implementation, a method comprises receiving scan data of a dental arch of a patient, providing the scan data as input to an artificial intelligence (AI) model trained to provide one or more indications of a predicted dental condition, receiving, as output from the AI model, the one or more indications of the predicted dental condition, and providing, to a user device, the one or more indications of the predicted dental condition.

A one hundred sixteenth implementation may further extend the one hundred fifteenth implementation. In the one hundred sixteenth implementation, the predicted dental condition comprises at least one of tooth wear, gum recession, gingival inflammation, chipped tooth, bruxism-related damage, gastroesophageal reflux disease (GERD) effects, or a missing attachment.

A one hundred seventeenth implementation may further extend the one hundred fifteenth through one hundred sixteenth implementations. In the one hundred seventeenth implementation, the scan data comprises color data, and the predicted dental condition comprises at least one of tooth staining, loss of vitality, poor oral care indicator, fluorosis, tooth decay, gingivitis, or a soft tissue lesion.

A one hundred eighteenth implementation may further extend the one hundred fifteenth through one hundred seventeenth implementations. In the one hundred eighteenth implementation, the method further comprises identifying a training dataset comprising a plurality of scan data, wherein the plurality of scan data comprises labels indicating a future dental condition, and training the AI model using the training dataset to provide the one or more indications of the predicted dental condition.

A one hundred nineteenth implementation may further extend the one hundred fifteenth through one hundred eighteenth implementations. In the one hundred nineteenth implementation, an indication of the one or more indications of the predicted dental condition comprises an estimated amount of progression of the predicted dental condition.

A one hundred twentieth implementation may further extend the one hundred fifteenth through one hundred nineteenth implementations. In the one hundred twentieth implementation, the indication of the one or more indications of the predicted dental condition comprises the estimated amount of the progression of the predicted dental condition for a particular time period.

A one hundred twenty-first implementation may further extend the one hundred fifteenth through one hundred twentieth implementations. In the one hundred twenty-first implementation, the method further comprises segmenting the scan data into individual teeth before providing the scan data to the AI model, wherein the one or more indications of the predicted dental condition correspond to at least one of a particular tooth or a particular gingival region.

A one hundred twenty-second implementation may further extend the one hundred fifteenth through one hundred twenty-first implementations. In the one hundred twenty-second implementation, each of the one or more indications comprises an estimated amount of the progression of the predicted dental condition, and the method further comprises identifying an indication of the one or more indications of the predicted dental condition for the particular tooth, wherein the indication corresponds to a highest estimated amount of the progression of the predicted dental condition, and providing, to the user device, the identified indication of the predicted dental condition for the particular tooth or the particular gingival region.

A one hundred twenty-third implementation may further extend the one hundred fifteenth through one hundred twenty-second implementations. In the one hundred twenty-third implementation, the method further comprises generating a heat map comprising the one or more indications of the predicted dental condition, wherein the heat map reflects an estimated amount of the progression of the predicted dental condition of the one or more indications, and providing, for display on the user device, the heat map.

A one hundred twenty-fourth implementation may further extend the one hundred fifteenth through one hundred twenty-third implementations. In the one hundred twenty-fourth implementation, the method further comprises aggregating the estimated amount of the progression of the predicted dental condition of the one or more indications for the particular tooth or for the particular gingival region.

A one hundred twenty-fifth implementation may further extend the one hundred fifteenth through one hundred twenty-fourth implementations. In the one hundred twenty-fifth implementation, the AI model is a generative AI model that is trained to generate a three-dimensional mesh to display the one or more indications of the predicted dental condition on corresponding identified teeth or gingival regions.

A one hundred twenty-sixth implementation may further extend the one hundred fifteenth through one hundred twenty-fifth implementations. In the one hundred twenty-sixth implementation, the scan data comprises at least one of: one or more three-dimensional mesh, one or more two-dimensional scan, or one or more occlusal maps.

A one hundred twenty-seventh implementation may further extend the one hundred fifteenth through one hundred twenty-sixth implementations. In the one hundred twenty-seventh implementation, the scan data comprises data from multiple bite positions of the dental arch of the patient.

A one hundred twenty-eighth implementation may further extend the one hundred fifteenth through one hundred twenty-seventh implementations. In the one hundred twenty-eighth implementation, the scan data comprises information on opposing teeth.

A one hundred twenty-ninth implementation may further extend the one hundred fifteenth through one hundred twenty-eighth implementations. In the one hundred twenty-ninth implementation, the method further comprises providing, as further input to the AI model, a time frame for the predicted dental condition, wherein the output from the AI model corresponds to the time frame.

A one hundred thirtieth implementation may further extend the one hundred fifteenth through one hundred twenty-ninth implementations. In the one hundred thirtieth implementation, the time frame is received from the user device.

A one hundred thirty-first implementation may further extend the one hundred fifteenth through one hundred thirtieth implementations. In the one hundred thirty-first implementation, an indication of the one or more indications of the predicted dental condition comprises a range of values corresponding to a severity range of a dental condition.

A one hundred thirty-second implementation may further extend the one hundred fifteenth through one hundred thirty-first implementations. In the one hundred thirty-second implementation, the method further comprises receiving, from the user device, a first indication of a severity level, and providing, for display on the user device, a first value of the range of values, wherein the first value corresponds to the first indication of the severity level, and wherein the first value of the range of values reflects the severity level of the predicted dental condition.

A one hundred thirty-third implementation may further extend the one hundred fifteenth through one hundred thirty-second implementations. In the one hundred thirty-third implementation, the method further comprises receiving updated scan data of the dental arch of the patient, wherein a timing of the updated scan data corresponds to the one or more indications of the predicted dental condition provided by the AI model, comparing the one or more indications of the predicted dental condition to the updated scan data, determining, based on the comparison, one or more inconsistencies between the updated scan data and the one or more indications, and determining a prognosis factor corresponding the one or more inconsistencies.

A one hundred thirty-fourth implementation may further extend the one hundred fifteenth through one hundred thirty-third implementations. In the one hundred thirty-fourth implementation, the method further comprises providing, as further input to the AI model, the updated scan data, receiving, as further output from the AI model, one or more updated indications of the predicted dental condition, applying the prognosis factor corresponding to the one or more inconsistencies to the one or more updated indications of the predicted dental condition, and providing, to the user device, the one or more updated indications of the predicted dental condition.

In a one hundred thirty-fifth implementation, a system comprises one or more computing devices each comprising a memory and one or more processing devices, wherein the one or more computing devices are configured to receive scan data of a dental arch of a patient, provide the scan data as input to an artificial intelligence (AI) model trained to provide one or more indications of a predicted dental condition, receive, as output from the AI model, the one or more indications of the predicted dental condition, and provide, to a user device, the one or more indications of the predicted dental condition.

A one hundred thirty-sixth implementation may further extend the one hundred thirty-fifth implementation. In the one hundred thirty-sixth implementation, the predicted dental condition comprises at least one of tooth wear, gum recession, gingival inflammation, chipped tooth, bruxism-related damage, gastroesophageal reflux disease (GERD) effects, or a missing attachment.

A one hundred thirty-seventh implementation may further extend the one hundred thirty-fifth through one hundred thirty-sixth implementations. In the one hundred thirty-seventh implementation, the scan data comprises color data, and the predicted dental condition comprises at least one of tooth staining, loss of vitality, poor oral care indicator, fluorosis, tooth decay, gingivitis, or a soft tissue lesion.

A one hundred thirty-eighth implementation may further extend the one hundred thirty-fifth through one hundred thirty-seventh implementations. In the one hundred thirty-eighth implementation, the one or more computing devices are further configured to identify a training dataset comprising a plurality of scan data, wherein the plurality of scan data comprises labels indicating a future dental condition, and train the AI model using the training dataset to provide the one or more indications of the predicted dental condition.

A one hundred thirty-ninth implementation may further extend the one hundred thirty-fifth through one hundred thirty-eighth implementations. In the one hundred thirty-ninth implementation, an indication of the one or more indications of the predicted dental condition comprises an estimated amount of progression of the predicted dental condition.

A one hundred fortieth implementation may further extend the one hundred thirty-fifth through one hundred thirty-ninth implementations. In the one hundred fortieth implementation, the indication of the one or more indications of the predicted dental condition comprises the estimated amount of the progression of the predicted dental condition for a particular time period.

A one hundred forty-first implementation may further extend the one hundred thirty-fifth through one hundred fortieth implementations. In the one hundred forty-first implementation, the one or more computing devices are further configured to segment the scan data into individual teeth before providing the scan data to the AI model, wherein the one or more indications of the predicted dental condition correspond to at least one of a particular tooth or a particular gingival region.

A one hundred forty-second implementation may further extend the one hundred thirty-fifth through one hundred forty-first implementations. In the one hundred forty-second implementation, each of the one or more indications comprises an estimated amount of the progression of the predicted dental condition, and the one or more computing devices are further configured to identify an indication of the one or more indications of the predicted dental condition for the particular tooth, wherein the indication corresponds to a highest estimated amount of the progression of the predicted dental condition, and provide, to the user device, the identified indication of the predicted dental condition for the particular tooth or the particular gingival region.

A one hundred forty-third implementation may further extend the one hundred thirty-fifth through one hundred forty-second implementations. In the one hundred forty-third implementation, the one or more computing devices are further configured to generate a heat map comprising the one or more indications of the predicted dental condition, wherein the heat map reflects an estimated amount of the progression of the predicted dental condition of the one or more indications, and provide, for display on the user device, the heat map.

A one hundred forty-fourth implementation may further extend the one hundred thirty-fifth through one hundred forty-third implementations. In the one hundred forty-fourth implementation, the one or more computing devices are further configured to aggregate the estimated amount of the progression of the predicted dental condition of the one or more indications for the particular tooth or for the particular gingival region.

A one hundred forty-fifth implementation may further extend the one hundred thirty-fifth through one hundred forty-fourth implementations. In the one hundred forty-fifth implementation, the AI model is a generative AI model that is trained to generate a three-dimensional mesh to display the one or more indications of the predicted dental condition on corresponding identified teeth or gingival regions.

A one hundred forty-sixth implementation may further extend the one hundred thirty-fifth through one hundred forty-fifth implementations. In the one hundred forty-sixth implementation, the scan data comprises at least one of: one or more three-dimensional mesh, one or more two-dimensional scan, or one or more occlusal maps.

A one hundred forty-seventh implementation may further extend the one hundred thirty-fifth through one hundred forty-sixth implementations. In the one hundred forty-seventh implementation, the scan data comprises data from multiple bite positions of the dental arch of the patient.

A one hundred forty-eighth implementation may further extend the one hundred thirty-fifth through one hundred forty-seventh implementations. In the one hundred forty-eighth implementation, the scan data comprises information on opposing teeth.

A one hundred forty-ninth implementation may further extend the one hundred thirty-fifth through one hundred forty-eighth implementations. In the one hundred forty-ninth implementation, the one or more computing devices are further configured to provide, as further input to the AI model, a time frame for the predicted dental condition, wherein the output from the AI model corresponds to the time frame.

A one hundred fiftieth implementation may further extend the one hundred thirty-fifth through one hundred forty-ninth implementations. In the one hundred fiftieth implementation, the time frame is received from the user device.

A one hundred fifty-first implementation may further extend the one hundred thirty-fifth through one hundred fiftieth implementations. In the one hundred fifty-first implementation, an indication of the one or more indications of the predicted dental condition comprises a range of values corresponding to a severity range of a dental condition.

A one hundred fifty-second implementation may further extend the one hundred thirty-fifth through one hundred fifty-first implementations. In the one hundred fifty-second implementation, the one or more computing devices are further configured to receive, from the user device, a first indication of a severity level, and provide, for display on the user device, a first value of the range of values, wherein the first value corresponds to the first indication of the severity level, and wherein the first value of the range of values reflects the severity level of the predicted dental condition.

A one hundred fifty-third implementation may further extend the one hundred thirty-fifth through one hundred fifty-second implementations. In the one hundred fifty-third implementation, the one or more computing devices are further configured to receive updated scan data of the dental arch of the patient, wherein a timing of the updated scan data corresponds to the one or more indications of the predicted dental condition provided by the AI model, compare the one or more indications of the predicted dental condition to the updated scan data, determine, based on the comparison, one or more inconsistencies between the updated scan data and the one or more indications, and determine a prognosis factor corresponding the one or more inconsistencies.

A one hundred fifty-fourth implementation may further extend the one hundred thirty-fifth through one hundred fifty-third implementations. In the one hundred fifty-fourth implementation, the one or more computing devices are further configured to provide, as further input to the AI model, the updated scan data, receive, as further output from the AI model, one or more updated indications of the predicted dental condition, apply the prognosis factor corresponding to the one or more inconsistencies to the one or more updated indications of the predicted dental condition, and provide, to the user device, the one or more updated indications of the predicted dental condition.

In a one hundred fifty-fifth implementation, a non-transitory computer-readable storage medium comprises instructions that, when executed by a processing device, cause the processing device to receive scan data of a dental arch of a patient, provide the scan data as input to an artificial intelligence (AI) model trained to provide one or more indications of a predicted dental condition, receive, as output from the AI model, the one or more indications of the predicted dental condition, and provide, to a user device, the one or more indications of the predicted dental condition.

A one hundred fifty-sixth implementation may further extend the one hundred fifty-fifth implementation. In the one hundred fifty-sixth implementation, the predicted dental condition comprises at least one of tooth wear, gum recession, gingival inflammation, chipped tooth, bruxism-related damage, gastroesophageal reflux disease (GERD) effects, or a missing attachment.

A one hundred fifty-seventh implementation may further extend the one hundred fifty-fifth through one hundred fifty-sixth implementations. In the one hundred fifty-seventh implementation, the scan data comprises color data, and the predicted dental condition comprises at least one of tooth staining, loss of vitality, poor oral care indicator, fluorosis, tooth decay, gingivitis, or a soft tissue lesion.

A one hundred fifty-eighth implementation may further extend the one hundred fifty-fifth through one hundred fifty-seventh implementations. In the one hundred fifty-eighth implementation, the processing device is further to identify a training dataset comprising a plurality of scan data, wherein the plurality of scan data comprises labels indicating a future dental condition, and train the AI model using the training dataset to provide the one or more indications of the predicted dental condition.

A one hundred fifty-ninth implementation may further extend the one hundred fifty-fifth through one hundred fifty-eighth implementations. In the one hundred fifty-ninth implementation, an indication of the one or more indications of the predicted dental condition comprises an estimated amount of progression of the predicted dental condition.

A one hundred sixtieth implementation may further extend the one hundred fifty-fifth through one hundred fifty-ninth implementations. In the one hundred sixtieth implementation, the indication of the one or more indications of the predicted dental condition comprises the estimated amount of the progression of the predicted dental condition for a particular time period.

A one hundred sixty-first implementation may further extend the one hundred fifty-fifth through one hundred sixtieth implementations. In the one hundred sixty-first implementation, the processing device is further to segment the scan data into individual teeth before providing the scan data to the AI model, wherein the one or more indications of the predicted dental condition correspond to at least one of a particular tooth or a particular gingival region.

A one hundred sixty-second implementation may further extend the one hundred fifty-fifth through one hundred sixty-first implementations. In the one hundred sixty-second implementation, each of the one or more indications comprises an estimated amount of the progression of the predicted dental condition, and the processing device is further to identify an indication of the one or more indications of the predicted dental condition for the particular tooth, wherein the indication corresponds to a highest estimated amount of the progression of the predicted dental condition, and provide, to the user device, the identified indication of the predicted dental condition for the particular tooth or the particular gingival region.

A one hundred sixty-third implementation may further extend the one hundred fifty-fifth through one hundred sixty-second implementations. In the one hundred sixty-third implementation, the processing device is further to generate a heat map comprising the one or more indications of the predicted dental condition, wherein the heat map reflects an estimated amount of the progression of the predicted dental condition of the one or more indications, and provide, for display on the user device, the heat map.

A one hundred sixty-fourth implementation may further extend the one hundred fifty-fifth through one hundred sixty-third implementations. In the one hundred sixty-fourth implementation, the processing device is further to aggregate the estimated amount of the progression of the predicted dental condition of the one or more indications for the particular tooth or for the particular gingival region.

A one hundred sixty-fifth implementation may further extend the one hundred fifty-fifth through one hundred sixty-fourth implementations. In the one hundred sixty-fifth implementation, the AI model is a generative AI model that is trained to generate a three-dimensional mesh to display the one or more indications of the predicted dental condition on corresponding identified teeth or gingival regions.

A one hundred sixty-sixth implementation may further extend the one hundred fifty-fifth through one hundred sixty-fifth implementations. In the one hundred sixty-sixth implementation, the scan data comprises at least one of: one or more three-dimensional mesh, one or more two-dimensional scan, or one or more occlusal maps.

A one hundred sixty-seventh implementation may further extend the one hundred fifty-fifth through one hundred sixty-sixth implementations. In the one hundred sixty-seventh implementation, the scan data comprises data from multiple bite positions of the dental arch of the patient.

A one hundred sixty-eighth implementation may further extend the one hundred fifty-fifth through one hundred sixty-seventh implementations. In the one hundred sixty-eighth implementation, the scan data comprises information on opposing teeth.

A one hundred sixty-ninth implementation may further extend the one hundred fifty-fifth through one hundred sixty-eighth implementations. In the one hundred sixty-ninth implementation, the processing device is further to provide, as further input to the AI model, a time frame for the predicted dental condition, wherein the output from the AI model corresponds to the time frame.

A one hundred seventieth implementation may further extend the one hundred fifty-fifth through one hundred sixty-ninth implementations. In the one hundred seventieth implementation, the time frame is received from the user device.

A one hundred seventy-first implementation may further extend the one hundred fifty-fifth through one hundred seventieth implementations. In the one hundred seventy-first implementation, an indication of the one or more indications of the predicted dental condition comprises a range of values corresponding to a severity range of a dental condition.

A one hundred seventy-second implementation may further extend the one hundred fifty-fifth through one hundred seventy-first implementations. In the one hundred seventy-second implementation, the processing device is further to receive, from the user device, a first indication of a severity level, and provide, for display on the user device, a first value of the range of values, wherein the first value corresponds to the first indication of the severity level, and wherein the first value of the range of values reflects the severity level of the predicted dental condition.

A one hundred seventy-third implementation may further extend the one hundred fifty-fifth through one hundred seventy-second implementations. In the one hundred seventy-third implementation, the processing device is further to receive updated scan data of the dental arch of the patient, wherein a timing of the updated scan data corresponds to the one or more indications of the predicted dental condition provided by the AI model, compare the one or more indications of the predicted dental condition to the updated scan data, determine, based on the comparison, one or more inconsistencies between the updated scan data and the one or more indications, and determine a prognosis factor corresponding the one or more inconsistencies.

A one hundred seventy-fourth implementation may further extend the one hundred fifty-fifth through one hundred seventy-third implementations. In the one hundred seventy-fourth implementation, the processing device is further to provide, as further input to the AI model, the updated scan data, receive, as further output from the AI model, one or more updated indications of the predicted dental condition, apply the prognosis factor corresponding to the one or more inconsistencies to the one or more updated indications of the predicted dental condition, and provide, to the user device, the one or more updated indications of the predicted dental condition.

In a one hundred seventy-fifth implementation, a system comprises a first computing device configured to provide intraoral scan data to a server device, and the server device, configured to receive the intraoral scan data, process the intraoral scan data using an artificial intelligence (AI) model, wherein the AI model is to output a value indicating a predicted dental condition, and provide, to the first computing device or a second computing device, an indication of the predicted dental condition based on the output provided by the AI model.

### BRIEF DESCRIPTION OF THE DRAWINGS

Aspects and embodiments of the present disclosure will be understood more fully from the detailed description given below and from the accompanying drawings of various aspects and embodiments of the disclosure, which, however, should not be taken to limit the disclosure to the specific aspects or embodiments, but are for explanation and understanding only.

FIG. 1A shows a block diagram of an example system for providing a dental condition prognosis, in accordance with some embodiments of the present disclosure.

FIG. 1B shows a block diagram of an example system for providing a dental condition prognosis, in accordance with some embodiments of the present disclosure.

FIG. 2 shows a block diagram of an example prognosis system, in accordance with some embodiments of the present disclosure.

FIG. 3 illustrates a flow diagram of an example method for generating training data by automatically labeling scan data with indications of a future dental condition, in accordance with some embodiments of the present disclosure.

FIG. 4 illustrates a flow diagram of an example method for providing indications of a predicted dental condition in a patient, in accordance with some embodiments of the present disclosure.

FIG. 5 illustrates workflows for training one or more artificial intelligence models to provide indications of a predicted dental condition, in accordance with some embodiments of the present disclosure.

FIG. 6 illustrates a block diagram of an example computing device, in accordance with some embodiments of the present disclosure.

### DETAILED DESCRIPTION

Described herein are embodiments of providing a dental condition prognosis. As used herein, “dental condition” or “progressive dental condition” refers to any condition affecting teeth, gingiva, or supporting oral structures that changes over time, including tooth wear, gingival recession, gingival inflammation, structural tooth changes, tissue discoloration, and/or other conditions visible through clinical examination and/or diagnostic imaging. Conventional methods of assessing dental conditions can involve a multifaceted approach that includes a doctor manually taking a patient history, performing a clinical examination, and analyzing diagnostic imaging. The patient history can include assessing the patient's risk factors, which can include identifying behavioral and/or lifestyle factors (such as teeth grinding or clenching habits), identifying dietary habits that may contribute to a particular dental condition (e.g., such as tooth wear), oral hygiene practices, medical conditions or medications, stress levels, etc. For example, during a clinical examination, a dental professional can perform a physical examination, which can include bite analysis, to identify existing tooth wear. In some cases, a dental professional can use a standardized system (such as a tooth wear index) to quantify wear levels and track changes over time. Diagnostic imaging can include, for example, dental x-rays, intraoral scans, cone beam computed tomography (CBCT) scans, and/or other types of imaging techniques. In some cases, dental professionals can compare multiple scans taken at different times to monitor progression by detecting changes in the teeth and/or mouth over time.

Thus, detecting a progressive dental condition can involve frequent checkups and monitoring to allow a dental professional to compare current wear with previous records. A patient may make multiple visits over time, allowing the dental professional to identify progression between visits. However, while a dental professional may be able to identify a future state of the dental condition by detecting a progression of the dental condition over multiple visits, it can be difficult or impossible for a dental professional to identify a potential future dental condition in a single visit (e.g., by analyzing a scan of a patient's dental arches performed during a single visit).

Aspects and implementations of the present disclosure address the above-noted challenges and deficiencies by providing a progressive dental condition prognosis system for identifying potential future progression of a dental condition based on image data of a patient's dental arch, such as a scan of a patient's dentition, and/or dental arches performed at a single point in time. As referred to herein, a progressive dental condition (sometimes referred to simply as a dental condition) can include tooth wear, gingival recession, gingival inflammation, chipped tooth, bruxism-related damage, gastroesophageal reflux disease (GERD) effects, missing attachment, tooth staining, loss of vitality, poor oral care indicators, fluorosis, tooth decay, gingivitis, soft tissue lesions, and other conditions that result in detectable changes in dental arch geometry, tissue appearance, or oral structure, for example. Tooth wear can include many types of mass tooth loss caused by, for example, attrition (e.g., gradual loss of tooth structured caused by tooth-to-tooth contact, such as mastication, grinding, or clenching), erosion (e.g., caused by chemical processes without the involvement of bacteria), abrasion (e.g., loss of tooth structure caused by mechanical forces from foreign objects or habits, e.g., physical wear caused by materials other than tooth contact), and/or abfraction (e.g., loss of tooth structure caused by mechanical stress at the cervical region of the tooth, often due to occlusal forces). Mass tooth loss can refer to tooth wear that is measured at a rate that is greater than expected for the patient's age. In some embodiments, the progressive dental condition prognosis system is referred to simply as the prognosis system. Although the detailed examples herein focus primarily on tooth wear for purposes of illustration, the prognosis system is equally applicable to all types of progressive dental conditions affecting teeth, gingiva, and/or supporting oral structures, and is not limited to tooth wear applications.

In some embodiments, the prognosis system described herein can implement an artificial intelligence (AI) model trained to identify one or more areas of potential future progressing dental conditions for a patient. The prognosis system can provide, as input to the trained AI model, image data (e.g., scan data, x-rays, CBCT scans, panoramic x-rays, etc.) of a patient. The scan data may be intraoral scan data generated by an intraoral scanner that can be generated from a scan (e.g., an intraoral scan) taken at a single point in time (e.g., during a single patient visit). The scan data can include, for example, a mesh segment from a 3D mesh or 3D point cloud generated from an intraoral scan, a height map for the mesh segment, surface normal directions, occlusal clearance data of teeth on one jaw relative to the other jaw, and/or other data corresponding to a scan of a patient. For example, the scan data may include near infrared (NIR) images of the patient dentition, color images of the patient dentition, images of the patient dentition generated using fluorescence imaging, and so on. The AI model can provide, as output, one or more indications of future dental condition(s) for the patient. In some embodiments, the one or more indications can be provided as a predicted delta map. The predicted delta map may provide differences between points on current teeth and corresponding points on predicted future teeth in embodiments, and/or differences between points on current gingiva and corresponding points of predicted future gingiva. In some embodiments, the prognosis system can normalize the delta map to a predetermined time period. In some embodiments, the prognosis system can display the indications of future dental condition(s) in a user interface of a user device (e.g., of a dental professional). In some embodiments, the AI model can generate a 3D model of the dental arches of the patient that includes the indications of predicted dental condition(s). The generated 3D model can be displayed in a user interface of the user device, to show a patient the effects of the identified potential future dental condition(s). A dental professional can use the generated 3D model to educate the patient on the predicted dental condition(s).

In some embodiments, the AI model can be trained using a training dataset that includes scan data that is labeled with indications of future wear and/or other progressing clinical indications. In some embodiments, the training dataset can be labeled automatically, as described further below and throughout. The scan data can include, for example, a 3D dental model and/or 2D projections of the 3D dental model. For example, during an intraoral scan of the patient's upper and lower arches, three-dimensional surface models of the individual arches can be created. These 3D models may be created for different time periods (e.g., different patient visits). An earlier in time 3D model (or images/projections generated therefrom) may be used as a training input and a later in time 3D model (or images/projections generated therefrom) may be used as a target output. Based on training of the AI model using a large enough dataset, the AI model may be trained to predict future dental condition(s) based on an input 3D model of a dental arch (or images/projections generated therefrom).

In some embodiments, aspects of the present disclosure provide an automatic labeling system that can automatically label scan data with indications of future dental condition(s). The automatic labeling system can compare two or more scans or 3D models of the same patient taken at different time periods (e.g., different patient visits), and identify inconsistencies between the scans or 3D models. In some embodiments, the automatic labeling system can segment scan data and/or 3D models into multiple sections. In some embodiments, each section can represent a particular tooth, or a section of the patient's mouth that correlates to a particular tooth. For example, segmenting an arch 3D model can include separating the individual teeth and other components within the 3D model into their own segmented, or isolated, model(s). Thus, the automatic labeling system can segment two or more scans or 3D models of a patient, where each scan/3D model is from a different point in time. In some embodiments, the segmentation data can supply local axes of the teeth. Based on the segmentation data, the automatic labeling system can identify occlusal areas of each tooth. For example, using the position and direction of the scanning apparatus (e.g., camera), the automatic labeling system can match each 2D image with the relevant data from the 3D mesh. The relevant data can include a mesh segment, distance map, surface normal direction, occlusal clearance to the opposing jaw, etc. The automatic labeling system can use this information to create a data set for each occlusal dental site.

The automatic labeling system can match each tooth from a first scan (e.g., taken at time T) to its occurrence in the second scan (e.g., taken at time T+1). In some embodiments, the tooth matching can be done using 3D registration and/or image registration. In some embodiments, 3D registration can include capturing 3D data of various points of a surface in multiple images (e.g., in multiple scans of the same patient), and registering the images by computing transformations between the images/scans. Registering each tooth can offset any tooth movement that may have occurred between scans of the patient's dentition. For example, if a patient has undergone orthodontic tooth movement between scans, registering each individual tooth (rather than a dental arch as a whole) can enable the automatic labeling system to identify changes in a particular tooth or gingival region caused by a particular progressive dental condition, rather than caused by the orthodontic treatment. The registration process can include determination of transformations which align a tooth in one image (from a first scan) with the same tooth in another image (from a second scan). In some embodiments, image registration involves identifying multiple points, point clouds, edges, corners, surface vectors, etc., in each image/scan of an image/scan pair, surface fitting to the points of each image/scan, and using local searches around points to match points of the two images/scans. In some embodiments, the automatic labeling system can assign the occlusal areas smaller weights during the registration process, so that registration is not affected by the progressive dental condition. That is, the automatic labeling system can identify the areas of occlusion for a particular tooth, and can give less weight to the areas of occlusion when registering images/scans from different patient visits.

In some embodiments, the automatic labeling system can calculate, for each segmented tooth or region, a delta map from the first scan to the second scan. In some embodiments, since the time elapsed between scans of a patient can vary, the automatic labeling system can normalize the delta map to a constant time period (e.g., one year). Thus, the delta map can represent the inconsistencies between the scans normalized to a predetermined time period. The automatic labeling system can analyze the inconsistencies, for example, by comparing the inconsistencies to threshold conditions. For example, the automatic labeling system can compare the size of an identified inconsistency to a size threshold, and/or can identify a cause of the identified inconsistency. If the size and/or the cause of the identified inconsistency satisfy a criterion (e.g., if the size exceeds the size threshold, and/or if the cause is on a list of predetermined causes), the automatic labeling system can add a label to the first scan data indicating the location of the future wear. The labeled scan data can then be used to train the AI model to identify potential future wear.

Embodiments described herein provide for a method and apparatus for predicting future dental condition(s) based on a single scan of the patient's dental arch or multiple scans generated at different times, resulting in an efficient prediction of a dental condition in a single dentist visit. Aspects of the present disclosure can provide for identification of potential future dental condition(s) before signs of the dental condition(s) are detected. Such early detection and diagnosis can lead to faster and early treatment, which can drastically reduce or eliminate some dental condition(s). Thus, aspects of the present disclosure can result in increased patient satisfaction and improved diagnosis and treatment of dental condition(s). Additionally, aspects of the present disclosure can result in a reduction in the use of computing resources to identify and detect dental condition(s), as the systems and methods described herein can identify potential dental condition(s) based on a scan data of the patient's dental arch from a single point in time, eliminating the need to generate and compare scan data at two different points in time to identify dental condition(s) in some embodiments.

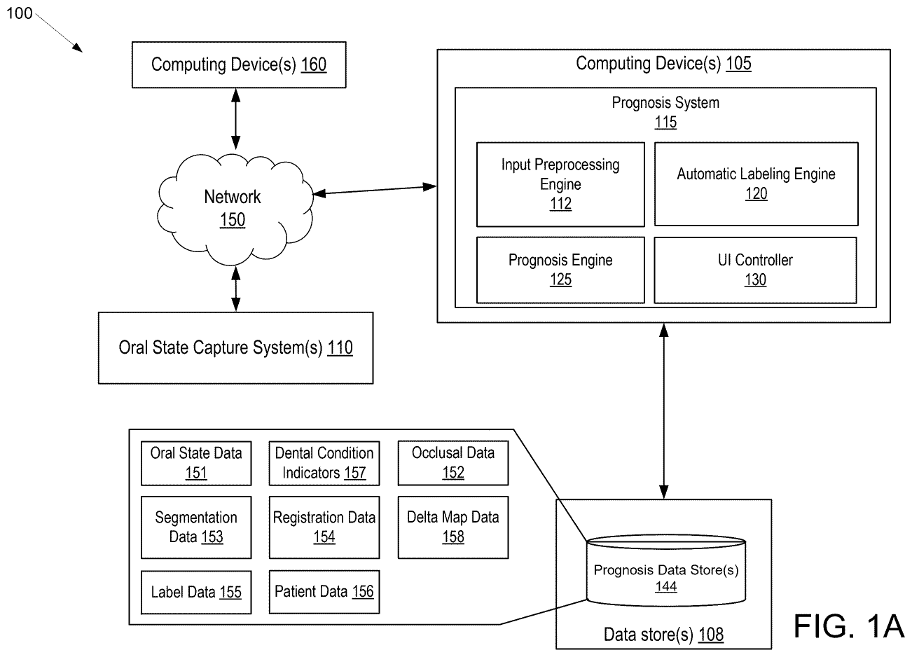

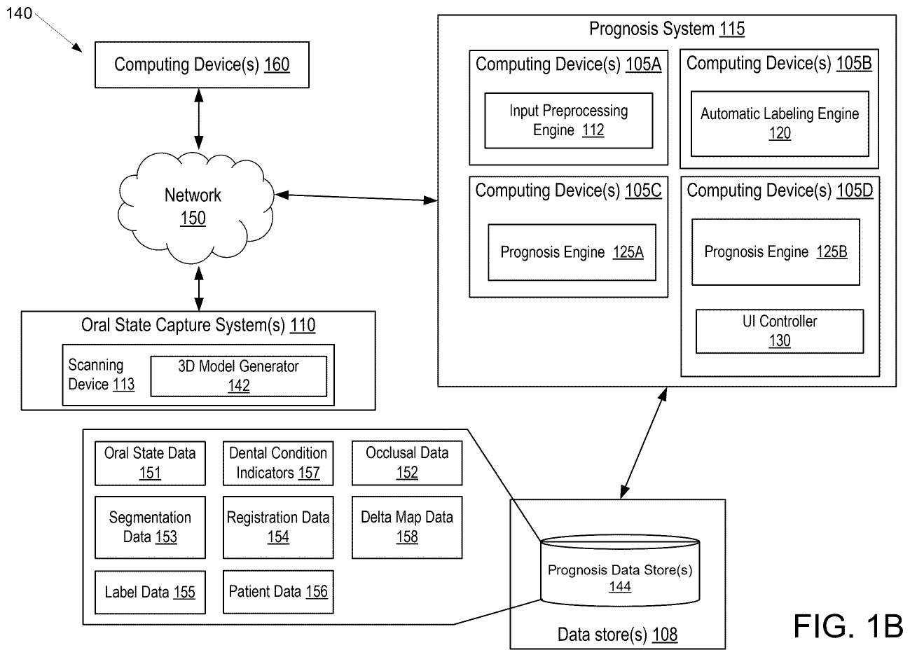

FIG. 1A illustrates a block diagram of an example system 100 for providing a dental condition prognosis, in accordance with some embodiments of the present disclosure. System 100 includes one or more computing device(s) 105 that may be coupled to one or more computing device(s) 160, one or more oral state capture system(s) 110, and/or one or more data store(s) 108.

Computing devices 105 and/or 160 may each include a processing device, memory, secondary storage, one or more input devices (e.g., such as a keyboard, mouse, tablet, and so on), one or more output devices (e.g., a display, a printer, etc.), and/or other hardware components. Computing device 105 may be connected to a data store 108 either directly or via a network (e.g., network 150). The network 150 may be a local area network (LAN), a public wide area network (WAN) (e.g., the Internet), a private WAN (e.g., an intranet), or a combination thereof. The computing device 105 may additionally or alternatively be connected to computing device(s) 160 and/or oral state capture systems 110 via a network 150, which may be a local area network (LAN), a public wide area network (WAN) (e.g., the Internet), a private WAN (e.g., an intranet), or a combination thereof. In some embodiments, oral state capture system(s) 110 connect to computing device(s) 105 directly via a wired or wireless connection.

Data store 108 may be an internal data store, or an external data store that is connected to computing device 105 directly or via a network. Examples of network data stores include a storage area network (SAN), a network attached storage (NAS), and a storage service provided by a cloud computing service provider. Data store 108 may include a file system, a database, or other data storage arrangement. In some embodiments, data store 108 can include one or more prognosis data store(s) 144. In some embodiments, the prognosis data store 144 can include oral state data 151, segmentation data 153, registration data 154, label data 155, patient data 156, dental condition indicators 157, delta map data 158, and/or occlusal data 152. In some embodiments, oral state data 151, segmentation data 153, registration data 154, label data 155, patient data 156, dental condition indicators 157, delta map data 158, and/or occlusal data 152 can reference a patient identifier.

In some embodiments, oral state data 151 can include image data corresponding to one or more scans (e.g., intraoral scan(s), CBCT scan(s), etc.), corresponding to one or more x-rays (e.g., panoramic x-rays), corresponding to one or more photographs (e.g., generated by a camera and/or a smart phone), corresponding to one or more sensors (e.g., generated by a compliance indicator), and/or other types of image data of a patient's dental arch(es), mouth, and/or face. In some embodiments, oral state data 151 can include data generated by the oral state capture system 110, as further described below. In some embodiments, oral state data 151 can be used to generate a virtual model (e.g., a virtual 2D model or a virtual 3D model) of a patient's dental arch(es). Each virtual model can reflect the condition of the dental arch at a particular point in time. Each virtual model can include a 3D surface and appearance properties mapped to each point of the 3D surface (e.g., mapped to points on the 3D surface as textures). In some embodiments, segmentation data 153 can include the segmented image data, e.g., as generated by input preprocessing engine 112, a segmented 3D model and/or segmented intraoral scans. Segmentation may be performed using one or more trained AI models (e.g., such as neural networks), which may perform instance segmentation and/or semantic segmentation in embodiments. In some embodiments, registration data 154 can include registration data generated by input preprocessing engine 112. In some embodiments, registration data 154 can store one or more transformation matrix that indicates the rotations, translations, and/or deformations that will cause one image/3D model (e.g., generated from a first scanning session) to corresponding to another image/3D model (e.g., generated from a second scanning session). In some embodiments, occlusal data 152 can include occlusal clearance between the jaws (e.g., the relationship between the maxillary (upper) teeth and mandibular (lower) teeth). In some embodiments, label data 155 can include labels generated by the automatic labeling engine 120. In some embodiments, dental condition indicators 157 can include indications of a dental condition, as identified by prognosis engine 125. In some embodiments, delta map data 158 can include the differences between first scan data/3D model(s) corresponding to a first intraoral scan taken at a first point in time, and a second scan data/3D model(s) corresponding to a second intraoral scan taken at a second point in time. In some embodiments, delta map data 158 can be generated by the automatic labeling engine 120.

In some embodiments, patient data 156 can include a patient chart (e.g., patient dental chart), which can include answers that the patient provided to a questionnaire. The questionnaire may be presented to the user in a clinical setting, e.g., by a clinician, technician, or medical professional. In some embodiments, the questionnaire may have been presented to the patient on a user device of the patient (e.g., one of computing devices 160). The patient's history may include, for example, past intraoral scans, 2D images and/or 3D models of the patient's dental arch(es) generated at various points in time, and/or prior assessments of other medical ailments and/or treatments. For example, patient data 156 can include a record of the patient's restorative work performed in the time between the two scans, an orthodontic treatment plan being implemented, and so on.

In some embodiments, oral state capture system(s) 110 can include an intraoral scanner, a microphone, a camera (e.g., capable of capturing images and/or video), a CBCT scanner, an x-ray machine and/or another imaging device, such as a CT scanner, an electronic compliance indicator (ECI) device or other dental appliance to be worn by a patient that includes a microphone, and/or optionally a computing device. The oral state capture system 110 can obtain image-based scans and/or images of a patient's dental arch(es), including the patient's dentition. In some embodiments, the intraoral scanner, microphone, camera, imaging device, ECI device, and/or processing device can be combined. For example, in some embodiments, a microphone can be built into the base of a scan wand of the intraoral scanner, which can be used to capture audio while a technician is using the intraoral scanner for other diagnostic capabilities (e.g., to perform intraoral scanning). In some embodiments, oral state capture system 110 includes a dental appliance such as an aligner, palatal expander, etc. that includes a microphone. As another example, in some embodiments, the processing device can be part of the intraoral scanner, CBCT scanner, and/or ECI device. In some embodiments, processing device can be part of a computing device 160, a computing device 105, and/or a separate device (not shown), and the oral state capture system 110 can send captured data (e.g., scan data, image data, audio data, and/or video data) for processing on a separate device. In one embodiment, oral state capture system 110 includes a patient or client device that can take 2D or 3D images of the patient's oral cavity in a non-clinical setting (e.g., at a patient's home).

In some embodiments, oral state capture system 110 may include a scanning system (e.g., CBCT scanner, intraoral scanner, and/or ECI device) that can perform scanning of the patient's mouth, jaw, head, and/or oral cavity. The scanning may be performed to generate scans of the patient's dental arch(es), which may be combined to generate one or more three dimensional (3D) models of a dentition and/or oral cavity. In some embodiments, oral state capture system 110 may include an imaging device, which may be a 2D or 3D imaging device, such as a digital camera, mobile phone, tablet computer, and so on. In some embodiments, the oral state capture system 110 may include a CBCT scanner to capture CBCT scans of the patient's dental arches. A CBCT scanner is a type of x-ray machine that uses a cone-shaped x-ray beam to capture data about the patient's anatomy. The CBCT scanner can generate multiple (e.g., 150-200) images from a variety of angles.

In some embodiments, oral state capture system 110 can be an intraoral scanning system comprising an intraoral scanner for obtaining intraoral scans (e.g., three-dimensional scans) of a patient's dentition and/or dental arches and an associated computing device (e.g., computing device 105) that may be connected to the intraoral scanner via a wired or wireless connection. In some embodiments, oral state capture system 110 can include a processing device, memory, secondary storage, one or more input devices, one or more output devices, and/or other hardware components, which can effectuate intraoral scanning. Additionally or alternatively, oral state capture system 110 can be connected to one or more computing device(s) (e.g., a computing device 160 and/or a computing device 105), which can be configured to effectuate intraoral scanning. In some embodiments, computing device 105, 160, and/or a computing device of oral state capture system 110 can include an intraoral scan application that processes intraoral scans generated by the intraoral scanner to generated 3D models of the patient's dentition, upper arch, and/or lower arch.

In some embodiments, an intraoral scanner of the oral state capture system 110 can include a probe (e.g., a hand held probe) for optically capturing three-dimensional structures. The intraoral scanner may be used to perform an intraoral scan of a patient's oral cavity. An intraoral scan application may run on computing device 105, 160, or on another computing device of the oral state capture system 110, and may communicate with the scanner to effectuate the intraoral scan. A result of the intraoral scan may be oral state data 151, which may include one or more sets of intraoral scans and/or intraoral images. Each intraoral scan may include a 3D point cloud generated from one or more two-dimensional (2D) and/or three-dimensional (3D) images (e.g., that may include depth information) of a portion of a dental site. In some embodiments, intraoral scans include x, y, and z information. In one embodiment, the intraoral scanner generates numerous discrete (i.e., individual) intraoral scans.

In some embodiments, sets of discrete intraoral scans are merged into a smaller set of blended intraoral scans, where each blended scan is a combination of multiple discrete scans. The oral state data 151 may include raw scans and/or blended scans, each of which may be referred to as intraoral scans (and in some instances as intraoral images). While scanning, the intraoral scanner may generate multiple (e.g., tens) of scans per second (referred to as raw scans). In some embodiments, in order to improve the quality of the data captured, a blending process may be used to combine a sequence of raw scans into a blended scan by some averaging process. Additionally, in some embodiments the intraoral scanner may generate many scans per second. This may be too much data to process using a artificial intelligence model in real time. Accordingly, groups of similar scans may be combined into the blended scans, and the blended scans may be input into one or more trained machine learning model. This may vastly reduce the computation resources used to process the intraoral scans without degrading quality. In one embodiment, each blended scan includes data from up to 20 raw scans, and further includes scans that differ by less than a threshold angular difference from one another and/or by less than a threshold positional difference from one another. Accordingly, some blended scans may include data from 20 scans, while other blended scans may include data from fewer than 20 scans. In one embodiment, the intraoral scan (which may be a blended scan) includes height values and intensity values for each pixel in the image.

In some embodiments, oral state data 151 may also include color 2D images and/or images of particular wavelengths (e.g., near-infrared (NIRI) images, infrared images, ultraviolet images, etc.) of a dental site. In some embodiments, intraoral scanner alternates between generation of 3D intraoral scans and one or more types of 2D intraoral images (e.g., color images, NIRI images, etc.) during scanning. For example, one or more 2D color images may be generated between generation of a fourth and fifth intraoral scan. For example, some scanners may include multiple image sensors that generate different 2D color images of different regions of a patient's dental arch concurrently. These 2D color images may be stitched together to form a single color representation of a larger field of view that includes a combination of the fields of view of the multiple image sensors.

In some embodiments, the oral state capture system 110 can include an ECI device. In some embodiments, the ECI device can be used to accurately monitor a patient's compliance to a prescribed aligner schedule. For instance, an aligner that is ECI-capable can have one or more sensors designed to detect temperature and/or proximity to a patient's tooth. The sensors can pair to a mobile phone, e.g., via a Bluetooth®-enabled “smart” aligner case, and can receive and/or transmit data between the mobile phone and the ECI. In some embodiments, the ECI device can include a pressure sensor that can measure pressure and can convert the measured physical pressure exerted on it into an electrical signal. The pressure sensor on the occlusal surface of the teeth can detect the occlusal force or biting pressure, which can be used to detect bruxism (grinding and/or clenching of the teeth). Such bruxism/grinding may cause tooth wear over time. The pressure sensor can include a sensing element that directly responds to pressure, a transducer that converts the physical change in the sensing element into an electrical signal, a signal conditioning component that can amplify, filter, and/or convert the signal into a digital signal, and/or an output component that can transmit the conditioned signal to a processing device. For example, the pressure sensor can be used to measure and analyze the forces exerted during various dental procedures and treatments, such as occlusal analysis, implantology, orthodontics, prosthodontics, and/or periodontology. In some embodiments, the pressure sensor can measure electrical activity recorded during execution of a sequence of actions (e.g., bruxism-related events such as teeth clenching and teeth grinding, etc., and/or bruxism-unrelated events such as swallowing, lightly nodding the head, lightly shaking the head, speaking, etc.). In some embodiments, the pressure sensor can record a time-averaged value during execution of a particular sequence of actions. The pressure sensor can detect, record, and/or transmit signals to a processing device (e.g., of the oral state capture system 110, and/or of computing device 105, 160). The pressure data (e.g., the detected signals) can indicate clenching or grinding of a patient. In some embodiments, the pressure sensor can be attached to a processing device in oral state capture system 110, or can be otherwise connected to a processing device in oral state capture system 110.

In some embodiments, oral state capture system 110 is connect to data store(s) 108 either directly or via network 150. In some embodiments, oral state capture system 110 transmits scan data (e.g., generated and/or captured by oral state capture system 110) to data store 108 for storage therein.

According to an example, a user (e.g., a practitioner) may subject a patient to intraoral scanning. In doing so, the user may apply an intraoral scanner to one or more patient intraoral locations. The scanning may be divided into one or more segments (also referred to as roles). As an example, the segments may include a lower dental arch of the patient, an upper dental arch of the patient, one or more preparation teeth of the patient (e.g., teeth of the patient to which a dental device such as a crown or other dental prosthetic will be applied), one or more teeth which are contacts of preparation teeth (e.g., teeth not themselves subject to a dental device but which are located next to one or more such teeth or which interface with one or more such teeth upon mouth closure), and/or patient bite (e.g., scanning performed with closure of the patient's mouth with the scan being directed towards an interface area of the patient's upper and lower teeth). Via such scanner application, the intraoral scanner may provide oral state data 151 to computing device 105 (or to another computing device of oral state capture system 110). The oral state data 151 may be provided in the form of intraoral scan data sets, each of which may include 2D intraoral images (e.g., color 2D images) and/or 3D intraoral scans of particular teeth and/or regions of an intraoral site. In one embodiment, separate intraoral scan data sets are created for the maxillary arch, for the mandibular arch, for a patient bite, and/or for each preparation tooth. Alternatively, a single large intraoral scan data set is generated (e.g., for a mandibular and/or maxillary arch). Intraoral scans may be provided from the intraoral scanner to the computing device 105 (or other computing device) in the form of one or more points (e.g., one or more pixels and/or groups of pixels). For instance, the intraoral scanner may provide an intraoral scan as one or more 3D point clouds. The intraoral scans may each comprise height information.

The manner in which the oral cavity of a patient is to be scanned may depend on the procedure to be applied thereto. For example, if an upper or lower denture is to be created, then a full scan of the mandibular or maxillary edentulous arches may be performed. In contrast, if a bridge is to be created, then just a portion of a total arch may be scanned which includes an edentulous region, the neighboring preparation teeth (e.g., abutment teeth) and the opposing arch and dentition. Alternatively, full scans of upper and/or lower dental arches may be performed if a bridge is to be created.

By way of non-limiting example, dental procedures may be broadly divided into prosthodontic (restorative) and orthodontic procedures, and then further subdivided into specific forms of these procedures. Additionally, dental procedures may include identification and treatment of gum disease, sleep apnea, and intraoral conditions such as malocclusions, temporomandibular joint disorder (TMD), gingival recession, tooth grinding, and so on. The term prosthodontic procedure refers, inter alia, to any procedure involving the oral cavity and directed to the design, manufacture or installation of a dental prosthesis at a dental site within the oral cavity (intraoral site), or a real or virtual model thereof, or directed to the design and preparation of the intraoral site to receive such a prosthesis. A prosthesis may include any restoration such as crowns, veneers, inlays, onlays, implants and bridges, for example, and any other artificial partial or complete denture. The term orthodontic procedure refers, inter alia, to any procedure involving the oral cavity and directed to the design, manufacture or installation of orthodontic elements at an intraoral site within the oral cavity, or a real or virtual model thereof, or directed to the design and preparation of the intraoral site to receive such orthodontic elements. These elements may be appliances including but not limited to brackets and wires, retainers, clear aligners, or functional appliances.

In embodiments, intraoral scanning may be performed on a patient's oral cavity during a visitation of a dental office. The intraoral scanning may be performed, for example, as part of a semi-annual or annual dental health checkup. The intraoral scanning may also be performed before, during and/or after one or more dental treatments, such as orthodontic treatment and/or prosthodontic treatment. The intraoral scanning may be a full or partial scan of the upper and/or lower dental arches, and may be performed in order to gather information for performing dental diagnostics, to generate a treatment plan, to determine progress of a treatment plan, and/or for other purposes. The oral state data 151 generated from the intraoral scanning may include 3D scan data, 2D color images, NIR (near infrared) and/or infrared images, and/or ultraviolet images, of all or a portion of the upper jaw and/or lower jaw. The oral state data 151 may further include one or more intraoral scans showing a relationship of the upper dental arch to the lower dental arch. These intraoral scans may be usable to determine a patient bite and/or to determine occlusal contact information for the patient. The patient bite may include determined relationships between teeth in the upper dental arch and teeth in the lower dental arch.

Intraoral scanners may work by moving the intraoral scanner inside a patient's mouth to capture all viewpoints of one or more tooth. During scanning, the intraoral scanner is calculating distances to solid surfaces in some embodiments. Each intraoral scan is overlapped algorithmically, or ‘stitched’, with the previous set of scans to generate a growing 3D surface. As such, each scan is associated with a rotation in space, or a projection, to how it fits into the 3D surface.

During intraoral scanning, an intraoral scan application (e.g., executing on computing device 105 or a computing device of oral state capture system 110) may register and stitch together two or more intraoral scans generated thus far from the intraoral scan sessions. In one embodiment, performing registration includes capturing 3D data of various points of a surface in multiple scans, and registering the scans by computing transformations between the scans. One or more 3D surfaces may be generated based on the registered and stitched together intraoral scans during the intraoral scanning. The one or more 3D surfaces may be output to a display so that a doctor or technician can view their scan progress thus far. As each new intraoral scan is captured and registered to previous intraoral scans and/or a 3D surface, the one or more 3D surfaces may be updated, and the updated 3D surface(s) may be output to the display. In embodiments, separate 3D surfaces are generated for the upper jaw and the lower jaw. This process may be performed in real time or near-real time to provide an updated view of the captured 3D surfaces during the intraoral scanning process.

When a scan session or a portion of a scan session associated with a particular scanning role (e.g., upper jaw role, lower jaw role, bite role, etc.) is complete (e.g., all scans for an intraoral site or dental site have been captured), the intraoral scan application may automatically generate a virtual 3D model of one or more scanned dental sites (e.g., of an upper jaw and a lower jaw). The final 3D model(s) may each be a set of 3D points and their connections with each other (i.e., a mesh). To generate a virtual 3D model, the intraoral scan application may register and stitch together the intraoral scans generated from the intraoral scan session that are associated with a particular scanning role. The registration performed at this stage may be more accurate than the registration performed during the capturing of the intraoral scans, and may take more time to complete than the registration performed during the capturing of the intraoral scans. In one embodiment, performing scan registration includes capturing 3D data of various points of a surface in multiple scans, and registering the scans by computing transformations between the scans. The 3D data may be projected into a 3D space of a 3D model to form a portion of the 3D model. The intraoral scans may be integrated into a common reference frame by applying appropriate transformations to points of each registered scan and projecting each scan into the 3D space.

In one embodiment, registration is performed for adjacent or overlapping intraoral scans (e.g., each successive frame of an intraoral video). Registration algorithms are carried out to register two adjacent or overlapping intraoral scans (e.g., two adjacent blended intraoral scans) and/or to register an intraoral scan with a 3D model, which essentially involves determination of the transformations which align one scan with the other scan and/or with the 3D model. Registration may involve identifying multiple points in each scan (e.g., point clouds) of a scan pair (or of a scan and the 3D model), surface fitting to the points, and using local searches around points to match points of the two scans (or of the scan and the 3D model). For example, the intraoral scan application may match points of one scan with the closest points interpolated on the surface of another scan, and iteratively minimize the distance between matched points. Other registration techniques may also be used.

The intraoral scan application may repeat registration for all intraoral scans of a sequence of intraoral scans to obtain transformations for each intraoral scan, to register each intraoral scan with previous intraoral scan(s) and/or with a common reference frame (e.g., with the 3D model). The intraoral scan application may integrate intraoral scans into a single virtual 3D model (or two virtual 3D models, one for each dental arch) by applying the appropriate determined transformations to each of the intraoral scans. Each transformation may include rotations about one to three axes and translations within one to three planes.

The generated virtual 3D model can include color information. In some embodiments, the oral state data 151 can include color information, e.g., from 2D color images captured during the scanning process. The oral state capture system 110 can use the color information to add color texture to the 3D model(s). Once virtual 3D model(s) of the patient's dental arches are generated, they may be stored in data store 108 as a portion of oral state data 151 in embodiments.

In some embodiments, the oral state capture system 110 can use the oral state data 151 (e.g., a 3D model generated from the scan data) to generate an occlosugram for the patient, which can represent the occlusions in the patient's dentition. An occlusion is the contact between teeth. An occlusogram can illustrate the occlusal clearance of one or more teeth of the patient. For example, the occlusogram can include an occlusal clearance color map that shows the contact relationship between the teeth on the patient's dental arches. The occlusogram can indicate portions of the teeth that have excessive force in the patient's occlusions, portions that have mild force in the patient's occlusions, and/or portions that have no occlusions. The occlusogram can be stored in occlusal data 152 of data store 108.

In some embodiments, computing device 105 is a desktop computer, a laptop computer, a server computer, etc., located at a doctor office. In some embodiments, computing device 105 is a server computing device (e.g., of a data center) that may be accessed from client devices (e.g., client devices of doctors, patients, etc.). In some embodiments, computing device 105 is a virtual machine. For example, computing device 105 may be a virtual machine that runs in a cloud computing environment.

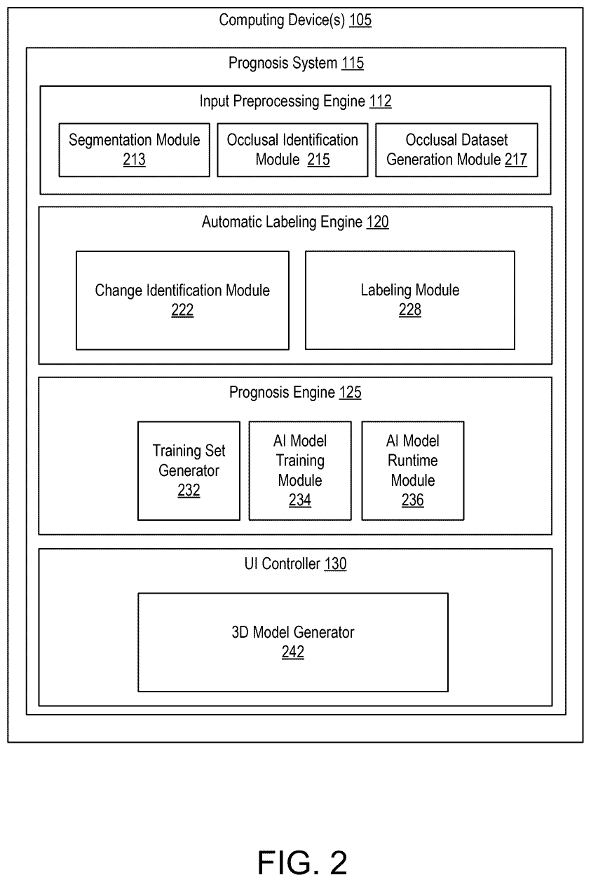

In some embodiments, computing device 105 includes a prognosis system 115. Prognosis system 115 may include software, hardware and/or firmware configured to perform one or more operations with respect to determining a dental condition prognosis. Prognosis system 115 can include an input preprocessing engine 112, an automatic labeling engine 120, a prognosis engine 125, and/or a user interface (UI) controller 130. The prognosis system 115 is further described with respect to FIG. 2.

In some embodiments, input preprocessing engine 112 can be a software program hosted by a device (e.g., computing device 105) to process oral state data (e.g., oral state data 151). Input preprocessing engine 112 may perform one or more operations on scan data 151 to prepare the oral state data 151 for analysis of potential future dental condition. Input preprocessing engine 112 may perform operations such as filtering, stabilizing, cropping, image enhancement (e.g., to sharpen an image), segmentation, registration, and/or other operations. In some embodiments, if oral state data 151 does not include a 3D model of a dental arch (e.g., includes 2D images or intraoral scans but no 3D models of dental arches), input preprocessing engine 112 may process the 2D images and/or intraoral scans to generate one or more 3D models (e.g., as discussed above). In some embodiments, 3D models may be generated from 2D images (e.g., such as those taken by a patient device such as a patient's mobile phone). In some embodiments, the input preprocessing engine 112 can project 3D oral state data 151 (e.g., a 3D model of a dental arch or intraoral scan) into 2D, e.g., using a mesh projection algorithm. Input preprocessing engine 112 can perform image segmentation that can then segment the 2D image data using 2D segmentation techniques. The 2D image data may be segmented into individual teeth, gingiva and/or other oral structures in embodiments. The resulting segmentation information can then be back-projected onto the 3D model or intraoral scan to apply segmentation information to the 3D model, and stored in segmentation data 153. Accordingly, in embodiments some or all of the individual teeth on a patient's upper and lower dental arches may be segmented and labeled on intraoral scans, 2D images and/or 3D models. Applicant hereby incorporates by reference the following application as if set forth fully here, as an example of 2D tooth segmentation: U.S. patent application Ser. No. 18/446,445 , filed Aug. 8, 2023, issued on Nov. 19, 2024, U.S. Pat. No. 12,144,701 B2. The input preprocessing engine 112 is further described with respect to FIG. 2.

In some embodiments, the automatic labeling engine 120 engine 120 can be a software program hosted by a device (e.g., computing device 105) to automatically generate labels to add to oral state data (e.g., oral state data 151). The automatically generated labels can indicate areas of future dental condition. In some embodiments, the automatic labeling engine 120 can be used for any clinical indication that is visible in scan geometry, including, for example, gingival recession, a chipped tooth, bruxism, Gastroesophageal Reflux Disease (GERD), gingival inflammation, a missing attachment, and/or other sources causing changes in the dental arch of the patient over time. The automatically generated labels (e.g., stored in label data 155) can be used to train an AI model to provide indications of future dental condition. The automatic labeling engine 120 is further described with respect to FIG. 2. In some embodiments, the automatic labeling engine 120 can be executed offline, as part of the data processing for the training the AI model(s).

In some embodiments, the prognosis engine 125 can be a software program hosted by a device (e.g., computing device 105) to provide one or more indications of potential future dental condition based on oral state data generated at a single point in time (e.g., during a visit to a dental professional) or multiple points in time. In some embodiments, prognosis engine 125 includes one or more trained AI models that have been trained to receive scan data and/or other oral state data (e.g., as output by a compliance device, a camera, etc.) as input, and to provide, as output, one or more indications of predicted dental condition(s), including for example indications of tooth wear, gingival recession, gingival inflammation, and/or other progressive clinical indications. The input data can correspond to oral state data 151. The one or more indications of predicted dental condition can be stored as dental condition indicators 157. The prognosis engine 125 is further described with respect to FIG. 2.

In some embodiments, patient data 156 can include a treatment plan for a current 3D mesh (e.g., oral state data 151). The treatment plan can include a predicted outcome of the patient's dental arches (e.g., tooth positioning) for a current stage of treatment and for after completion of the treatment plan. The treatment plan can include, for example, a series of dental aligners and/or a series of palatal expanders. The post-treatment outcome and/or intermediate outcome can be provided to the prognosis engine 125, which can provide one or more indications of predicted dental condition(s) for the post-treatment outcome and/or intermediate outcome of the treatment plan. This can enable a dental professional to show a patient that the treatment plan may result in reduced progression of dental condition(s) over time.

In some embodiments, the UI controller 130 can be a software program hosted by a device (e.g., computing device 105) to provide the one or more indications of predicted dental condition(s) (e.g., dental condition indicators 157) for display in a UI. In some embodiments, the UI controller 130 can display a 3D model of the patient's dental arch(es), including the indicators of predicted dental condition(s). In some embodiments, the UI controller 130 can display a current 3D model of the patient's dental arch(es), and include a list of potential dental condition indicators (and/or other progressing clinical findings) in the UI. In some embodiments, the dental condition indicators are displayed as an overlay over a current 3D model, and show a predicted future 3D state of the patient's teeth caused by the dental condition(s). For example, areas of current teeth that are predicted to be worn in the future may be shown using a visualization such as a different color, opacity, etc., than surrounding regions of teeth. In some embodiments, the areas affected by the dental condition(s) may be color coded or otherwise coded using different visualizations such that different visualizations are provided for different degrees of predicted dental condition(s). For example, excessive tooth wear may be shown in red, medium tooth wear may be shown in yellow or orange, and mild tooth wear may be shown in green. As another example, areas of current gums that are predicted to be affected by gingival recession and/or inflammation in the future may be shown using a visualization such as a different color, etc., than surrounding regions of gums. The dental condition indictors can be normalized to a predetermined time period (e.g., one year after the scan data used to identify the predicted dental condition indicators were generated) in embodiments. For example, the dental condition indicators may show an amount of change to teeth and/or gums at a particular future point in time. In some embodiments, a user may select a future point in time and the prognosis engine 125 may update a dental condition prediction for the designated point in time. The severity of the dental condition progression may depend at least in part on how far into the future dental condition is predicted for. The UI controller 130 can enable a user (e.g., a dental professional) to modify the predetermined time period, to display the dental condition indicators at various points in time.

In some embodiments, computing device(s) 160 can be or include a user device. In some embodiments, the user device 160 can be used by a dental professional (e.g., a doctor, a dentist, a hygienist, and/or a technician) to educate a patient regarding the patient's dental health. In some embodiments, the user device 160 can be used by a patient to review their dental health. The user device can be configured to output the indication(s) of predicted dental condition(s). The user device 160 can include a user interface (UI) to display the indications of predicted dental condition(s) (e.g., as provided by prognosis engine 125), patient data 156, and/or the images of oral state data 151 optionally overlaid with the segmentation data 154, and/or the dental condition indicators 157.

Applicant hereby incorporates by reference the following application as if set forth fully herein, as an example of an analysis and output for identifying tooth wear: U.S. Pat. Pub. No. 20220202295A1, published on Jun. 30, 2022, issued on Oct. 29, 2024, U.S. Pat. No. 12,127,814 B2.

FIG. 1B illustrates a block diagram of an example system 140 for providing dental condition prognosis, in accordance with some embodiments of the present disclosure. In some embodiments, the example system 140 can correspond to, and perform the same operations as the similarly labeled components of FIG. 1A. In some embodiments, oral state capture system 110 can include a scanning device 113 (e.g., an intraoral scanner) that is configured to generate scan data of a dental arch of a patient. The scanning device 113 can be operatively coupled to a computing device (e.g., one or more of computing devices 105A-D) configured to implement portions of the prognosis system 115. For example, the computing device can be configured to receive the scan data from the scanning device 113, provide the scan data as input to an AI model trained to output a value indicating predicted dental condition(s), and provide, to a user device (e.g., computing device 160), an indication of predicted dental condition(s) based on the output provided by the AI model. As another example, the computing device (e.g., a server device) can be configured to receive the scan data (e.g., from a computing device configured to provide intraoral scan data to the computing device), process the intraoral scan data using an AI model that is to output a value indicating predicted dental condition(s), and provide, to the computing device or to a second computing device (e.g., computing device 160) an indication of predicted dental condition(s) based on the output provided by the AI model.

In some embodiments, the prognosis system 115 can be spread among multiple computing devices 105A-D. Computing device 105A can execute the input preprocessing engine 112, and can perform the operations of input preprocessing engine 112 of FIG. 1A. In some embodiments, the input processing engine 112 can be executed offline, and the output of the preprocessing engine 112 can be stored in data store 114.

In some embodiments, computing device 105B can execute automatic labeling engine 120, which can perform the same operations of automatic labeling engine 120 of FIG. 1A. In some embodiments, automatic labeling engine 120 can be executed offline, as preprocessing for the training of prognosis engine 125. The output of the automatic labeling engine 120 can be stored data store 144 (e.g., as label data 155).

In some embodiments, computing device 105C can execute a first instance of prognosis engine 125A. The first instance of prognosis engine 125A can train AI model of prognosis engine 125. For example, as further described with respect to FIG. 2, computing device 105C can execute the training set generator 232 and/or the AI model training module 234. In some embodiments, the first instance of prognosis engine 125A can be executed offline, as preprocessing for creating the AI model. The trained AI model can be stored in data store 144.

In some embodiments, computing device 105D can execute a second instance of prognosis engine 125B. The second instance of prognosis engine 125B can run the AI model of prognosis engine. Computing device 105D can execute the AI model online multiple times, e.g., using new patient data (e.g., received from computing device 160, oral state capture system 110, and/or from another source). In some embodiments, as further described with respect to FIG. 2, computing device 105D can execute the AI model runtime module 236 of FIG. 2.

In some embodiments, oral state capture system 110 can include a scanning device 113. In some embodiments, the scanning device 113 can include a 3D model generator 142 that can generate the 3D models of patient's teeth, e.g., using the data stored in prognosis data store(s) 144.

FIG. 2 illustrates a diagram of an example prognosis system 115, in accordance with some embodiments of the present disclosure. In some embodiments, prognosis system 115 can include an input preprocessing engine 112, an automatic labeling engine 120, a prognosis engine 125, and/or a UI controller 130. In some embodiments, input preprocessing engine 112, automatic labeling engine 120, prognosis engine 125, and/or UI controller 130 can perform the same functions as input preprocessing engine 112, automatic labeling engine 120, prognosis engine 125, and/or UI controller 130 described with respect to FIG. 1A-B.

Input preprocessing engine 112 can be processing logic hosted by a device (e.g., computing device 105) to preprocess received data (e.g., oral state data 151 of FIG. 1A-B). Input preprocessing engine 112 can include a segmentation engine 213, an occlusal identification module 215, and/or a occlusal dataset generation module 217.

In some embodiments, segmentation module 213 segments scan data (e.g., 2D images, intraoral scans, 3D models, etc., of oral state data 151 of FIG. 1A-B) into features/objects, such as individual teeth (including tooth number), or areas of the patient's mouth that correspond to individual teeth (e.g., the gum around a missing tooth). In some embodiments, the segmentation module 213 can receive oral state data 151 and/or other patient data of or associated with a patient's dental arch(es). In some embodiments, the input preprocessing engine 112 can convert the image oral state data 151 into a 3D model, e.g., using sparse voxel segmentation, mesh segmentation, or point-based segmentation. The segmentation module 213 can include a trained machine learning model that takes scan data as input, and outputs segmentation data indicating the features (e.g., tooth number). Generated segmentation information may be stored as segmentation data 153 of FIG. 1A-B, in embodiments.

In some embodiments, segmentation module 213 is, or includes, a trained machine learning model that has been trained to perform semantic segmentation and/or instance segmentation of oral structures (e.g., to determine sizes, shapes, locations, tooth numbers, etc. of individual teeth, gingiva, etc.). Applicant hereby incorporates by reference the following application as if set forth fully here, as an example of a machine learning dental segmentation system and method, and training of such a machine learning segmentation system: U.S. Pat. Pub. No. 20210196434A1, published on Jul. 1, 2021, issued on Feb. 20, 2024, U.S. Pat. No. 11,903,793 B2.

In some embodiments, segmentation module 213 can output segmentation information for the oral state data 151, and can store the segmentation information in segmentation data 153. In some embodiments, segmentation module 213 can be or include, for example, a trained machine learning model such as a convolutional neural network (CNN) trained to classify pixels or regions of input images into different classes. This can include performing point-level classification (e.g., pixel-level classification or voxel-level classification) of different types of features and/or objects of subjects of images. The different features and/or objects may include, for example, individual teeth, regions of the dental arch corresponding to individual teeth, gingiva, etc. The trained machine learning model of segmentation module 213 may output one or more masks, each of which may have a same resolution as an input image. The mask or masks may include a different identifier for each identified feature or object, and may assign the identifiers on a pixel-level or patch-level basis. In one embodiment, different masks are generated for one or more different classes of features and/or objects and/or for each instance of a feature and/or object. In one embodiment, a single mask or map includes segmentation information for all identified classes of features and/or objects. Some types of features are location-specific features and are represented in one or more masks.

In some embodiments, segmentation module 213 can perform one or more processing and/or computer vision techniques or operations to extract segmentation information from images (e.g., oral state data 151). Such image processing and/or computer vision techniques may or may not include the use of trained machine learning models. Accordingly, in some embodiments, segmentation module 213 does not include a machine learning module.

In some embodiments, occlusal identification module 215 can identify occlusal areas of each segment (e.g., each tooth). Occlusal identification module 215 can generate an occlusion map based on occlusion contacts identified using digital models of the patient's upper and lower arches. The occlusal map can include the location and degree of contact between the teeth of the patient's upper arch and the teeth of the patient's lower arch. The occlusal map can include information such as the distance between a location on a surface of a tooth of one of the patient's upper or lower arch and a surface of the opposing tooth on the other one of the patient's upper or lower arch. The occlusal map can be stored in occlusal data 152 of FIG. 1A-B. Applicant hereby incorporates by reference the following application as if set forth fully here, as an example of capturing true bite and occlusion contacts: U.S. Pat. Pub. No. 20220323190A1, published on Oct. 13, 2022.

In some embodiments, occlusal dataset generation module 217 can generate, using the occlusal data 152, a dataset for each occlusal site. In some embodiments, occlusal dataset generation module 217 can match, for each 2D image or intraoral scan, the relevant data from the mesh. The relevant data can include, for example, a mesh segment, a distance map, a surface normal direction, an occlusal clearance to the opposite jaw, etc. The occlusal dataset can include the relevant information for each occlusal site.

Input preprocessing engine 112 can preprocess scan data from scans performed at various point in time. That is, input preprocessing engine 112 can perform the preprocessing operations described with respect to segmentation module 213, occlusal identification module 215, and/or occlusal dataset generation module 217 on scan data corresponding to a first scan performed at a first point in time, and on scan data corresponding to a second scan performed at a second point in time (e.g., six months later).

Automatic labeling engine 120 may process intraoral scan data to apply dental condition labels to such scan data and to generate training data that can be used to train a machine learning model to predict future dental condition(s) based on input scan data. In some embodiments, automatic labeling engine 120 can include processing logic hosted by a device (e.g., computing device 105) to automatically add labels to oral state data (e.g., oral state data 151, optionally processed by input preprocessing engine 112). In some embodiments, automatic labeling engine 120 can be executed offline (e.g., at a single point in time), as a preprocessing for the training of AI mode(s), e.g., to generate labeled data that forms the training dataset to train AI model(s). Automatic labeling engine 120 can include change identification module 222, and/or a labeling module 228.

In some embodiments, change identification module 222 can identify changes that have occurred in a dental arch over time. In some embodiments, change identification module 222 can match each tooth from first scan data taken at a first time to its occurrence in second scan data taken at a second time. Change identification module 222 can identify each segment (e.g., each tooth) in scan data corresponding to the first scan performed at a first point in time to the corresponding segment (e.g., tooth) in scan data corresponding to the second scan performed at a second point in time (e.g., six months later). In some embodiments, change identification module 222 can match each segment from the various scans using registration (e.g., 3D registration). Change identification module 222 may determine past dental condition(s) that has occurred in embodiments, which may be used to improve a prediction of future dental condition(s) in embodiments.

In some embodiments, change identification module 222 can perform 3D registration by computing transformations between the images (e.g., between a first image representing a first scan performed at a first point in time and a second image representing a second scan performed at a second point in time). In some embodiments, performing image registration includes capturing 3D data of various points of a surface in multiple images (views from a camera), and registering the images by computing transformations between the images. In some embodiments, during the registration process, change identification module 222 can assign smaller weights to the occlusal areas (e.g., as identified by occlusal identification module 215), so that the registration is not affected by a particular dental condition (e.g., tooth wear).

In some embodiments, change identification module 226 can compare the spatial information and/or appearance of the first representation of a tooth from the first scan data (generated by the first scan) to a second representation of the same tooth from the second scan data (generated by the second scan). In some embodiments, based on the spatial information and/or appearance comparisons, change identification module 226 can generate, for each tooth, a delta map that represents the changes between the first scan data corresponding to a scan performed at a first point in time, and the second scan data corresponding to a scan performed at a second point in time. The delta map represents the differences in each tooth between the first scan and the second scan, representing a dental condition that has already occurred.

In some embodiments, change identification module 226 can normalize the delta map to a particular time period. The time period between the first scan and the second scan may vary between patients. To account for the variation in time, change identification module 226 can normalize the differences in the delta map to a predetermined particular time period. To normalize the differences in the delta map, change identification module 226 can scale the difference values in the delta map so that they correspond to the predetermined time period. For example, change identification module 226 can determine the time difference between when the two scans were performed, compute a scaling factor based on the ratio of the predetermined time period to the actual time difference, and multiply each value in the delta map by the scaling factor. The normalization approach can vary based on the type of data stored in the delta map (e.g., pixel intensity, feature-based differences, etc.). Note that other normalization approaches can be used to normalize the delta map to a predetermined time period. In some embodiments, change identification module 226 can store the delta map in delta map data 158 of data store 108 of FIG. 1A-B.

Applicant hereby incorporates by reference the following application as if set forth fully here, as an example of identifying and classifying changes that have occurred in a dental arch over time, including registering image data of a dental arch: U.S. Pat. Pub. No. 20180235437A1, published on Aug. 23, 2018, issued on Dec. 10, 2019, U.S. Pat. No. 10,499,793 B2.

In some embodiments, labeling module 228 can generate labels (e.g., indicators) corresponding to identified area(s) affected by dental condition(s). The labeling module 228 can identify potential area(s) affected by dental condition(s) in the delta map (e.g., generated by change identification module 226). The labeling module 228 can compare each delta in the delta map to a threshold to determine whether the delta represents the dental condition. For example, the delta map can indicate a size of a change in the surface of the tooth, and the labeling module 228 can compare the size of the change to size threshold. If the size of the change satisfies a criterion (e.g., exceeds or the size threshold), the labeling module 228 can generate and/or add an indicator to the first scan data to indicate the future dental condition. The labeling module 228 can associate the label (or indicator) with the tooth, e.g., referenced by the tooth number.

In some embodiments, the labeling module 228 can identify a cause of a change of a tooth represented in the delta map. In some embodiments, the labeling module 228 can access the patient data (e.g., patient data 156 of FIG. 1A-B) to identify a cause. For example, the patient data can indicate that the patient has undergone restorative work in the time period between the two scans. The labeling module 228 can identify which teeth were affected by the restorative work. If the delta map includes a change in one of the teeth that was affected by the restorative work performed in the time period between the two scans, the labeling module 228 can determine that the cause of the change is due to the restorative work. The labeling module 228 can compare the identified cause to a list of causes to determine whether to add a label (or indicator) to the first scan to indicate the future dental condition. In some embodiments, the list of causes can include causes that cause a change in a tooth that is not related to a progressive dental conditions (e.g., restorative work). Thus, if the identified cause is on the list of causes not related to a progressive dental condition, the labeling module 228 can determine not to add a label (or indicator) to the first scan data corresponding to the change in the tooth. In some embodiments, the list of causes can include causes that cause change in a tooth that is related to progressive dental conditions, in which can the labeling module 228 can determine to add a label (or indicator) to the first scan data corresponding to the change in the tooth if the cause is included in the list of causes.

In some embodiments, the labeling module 228 can store the label data (e.g., the indicators added to the first scan data) in label data 155. Each label (or indicator) can reference a particular tooth of a patient.

In some embodiments, a dental professional can manually review the delta map and/or the label data to confirm and/or reject the changes in the teeth between the first scan and the second scan as related to dental condition(s). In some embodiments, the dental professional can manually review the changes that satisfy a criterion (e.g., that are below a threshold size, and/or that are identify as being caused by something other than progressive dental condition(s)).

In some embodiments, prognosis engine 125 can be processing logic hosted by a device (e.g., computing device 105) to provide one or more indications of predicted dental condition(s) based on oral state data taken at a single point in time (e.g., oral state data 151, optionally processed by input preprocessing engine 112). Prognosis engine 125 can include a training set generator 232, an AI training module 234, and/or a an AI model runtime module 236. In some embodiments, the training set generator 232 and/or the AI model training module 234 can be executed offline as preprocessing for creating the AI algorithm. In some embodiments, the AI model runtime module 236 can implement the trained AI model multiple times, e.g., as new oral state data is received from an oral state capture system 110 of FIG. 1A-B.

In some embodiments, training set generator 232 can generate one or more sets of training data used to train one or more artificial intelligence (AI) models. In some embodiments, a training dataset can include oral state data (e.g., oral state data 151) from the occlusion direction. In some embodiments, a training dataset can include multiple datasets of oral state data (e.g., oral state data 151) from several view directions. The training dataset(s) can include a mesh segment of a 3D model corresponding to a particular tooth, height map for that mesh segment, surface normal directions, and/or an occlusal clearance measurement to the other jaw. This data can be generated and/or identified by input preprocessing engine 112.

In some embodiments, the training dataset(s) can include the oral state data (e.g., oral state data 151) corresponding to a first scan as well as the labels indicating future tooth wear and/or other progressing dental conditions (such as gingival recession, chipped tooth, bruxism, GERD, gingival inflammation, missing attachment, etc.), e.g., generated by automatic labeling engine 120. In some embodiments, the training dataset(s) can include the scan data corresponding to the first scan as well as the delta map indicating the differences between the first scan and the second scan. In some embodiments, the training dataset(s) can also include patient data, such as information on opposing tooth. For example, a hard material crown in the opposing jaw may cause faster tooth wear.

In some embodiments, AI model training module 234 can train one or more AI models using the training dataset(s) generated by training set generator 232. In some embodiments, the AI model can be a neural network, trained on a training dataset that includes the data prepared from the first scan and the second scan, the delta map, and/or the labeling data. In some embodiments, the training dataset can be stacked as 2D with multiple channels. The channels can correspond to color, NIRI, surface normal, occlusal distances, height map, etc. In some embodiments, the AI training module 234 can train the AI model(s) to output a predicted delta map, indicating the areas affected by predicted future dental condition(s). In some embodiments, the training module 234 can train the AI model(s) to output a predicted map, indicating area(s) of other progressing indications, such as gingival recession and/or gingival inflammation. The AI training module 234 can implement a loss function that is a simple sum of differences between the real delta map and the projected delta map. In some embodiments, the loss function can correspond to the center area of the delta map.

In some embodiments, AI model runtime module 236 can implement the trained AI model. The trained AI model can receive, as input, oral state data, such as scan data prepared from a scan (e.g., an intraoral scan), CBCT scan data, x-ray data, image data (e.g., photographs), and/or other types of oral state data, corresponding to a single point in time. The trained AI model can provide, as output, a predicted delta map including indications of predicted future tooth wear and/or other progressing dental condition(s) (e.g., related to gingival recession, gingival inflammation, etc.). In some embodiments, the scan data provided as input can include data for an occlusal site that is visible in multiple 2D images. Thus, the 2D predictions for each occlusal site can be aggregated (e.g., either a mean, or 90^{Th} percentile max). In some embodiments, the trained AI model can provide an indication of future wear that is not related to occlusal surfaces. For example, an indication of future tooth wear can be identified as being related to abrasion or abfraction.

In some embodiments, the AI training module 234 can compare the output of the trained AI model to oral state data (e.g., scan data data generated from a scan) corresponding to a time period that matches the prediction time period of the output. The AI training module 234 can identify differences between the oral state data and the output of the trained AI model, and can apply a prognosis factor corresponding to the identified differences to future outputs of the model for that particular patient and/or for a particular tooth. For example, if the output of the AI model predicted a 1 millimeter wear for a particular tooth at time T+6, but an actual scan of the patient indicates a 1.2 millimeter wear of the particular tooth at time T+6, the AI training module 234 can apply a factor to account for the difference to future executions of the trained AI model to account for the difference.

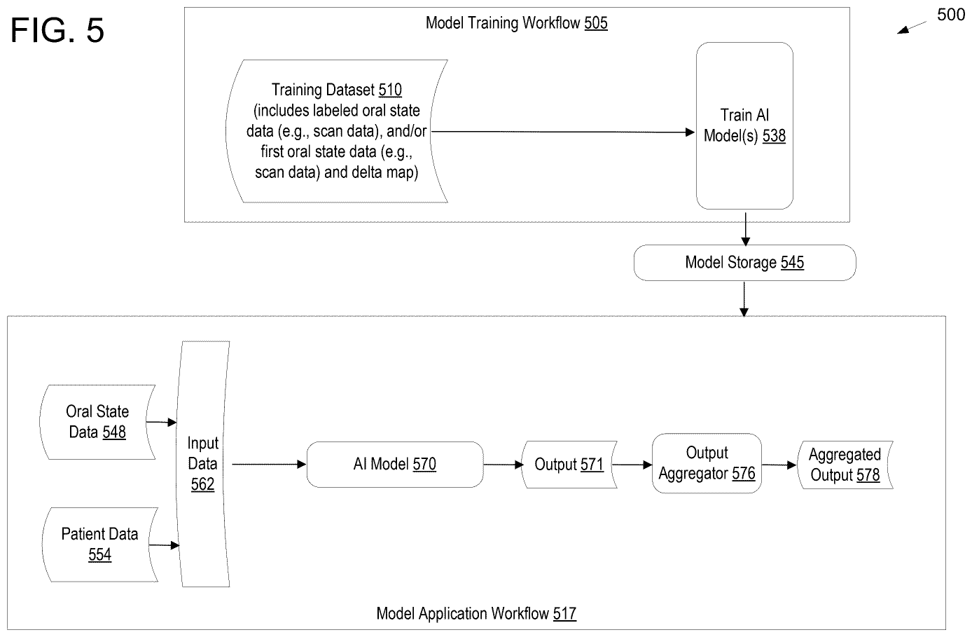

AI model training module 234 and AI model runtime module 236 are further described with respect to FIG. 5.

In some embodiments, UI controller 130 can be a software program hosted by a device (e.g., computing device 105) to generate a user interface for display on a user device (e.g., computing 105 and/or computing device 160 of FIG. 1A-B). The UI can display, for example, a list of the predicted dental condition(s) as provided by prognosis engine 125. For example, the UI can include a list of the dental condition indicators 157 (e.g., stored in data store 108 of FIG. 1A-B) that correspond to a particular patient or to a particular tooth of the patient. In some embodiments, the UI controller 130 can provide an interactive element in which the user can select one of the teeth in the list of dental condition indicators, and the UI can display a model (e.g., a 3D model) of the patient's dental arch(es), or portions thereof corresponding to the selected tooth. For example, the model displayed in the UI can rotate to show the selected tooth in the center of the screen.

In some embodiments, the UI can include an interactive element in which the user (e.g., the dental professional or the patient) can adjust the time of the prediction of future dental condition(s). The UI controller 130 can adjust the prediction based on the time of the prediction input by the user. For example, the prognosis engine 125 can output an indication of predicted dental condition for 6 months from the date of the scan data, and a user can change the prediction for a particular dental condition to 12 months. The UI controller can adjust the predicted dental condition progression based on the time period input by the user. For example, the UI controller can apply a normalizing factor to the predicted dental condition to adjust the prediction to the time period input by the user. As another example, the UI controller 130 can provide the time period input by the user to the trained AI model as additional input, and the trained AI model can output an updated dental condition prediction indicator corresponding to the time period input by the user.

In some embodiments, UI controller 130 can include a 3D model generator 242. In some embodiments, 3D model generator can include or communicate with a generative AI model that can generate a 3D model of a patient's dental arch(es) that includes the indicators of predicted dental condition(s). The generative AI model can automatically generate text, images, media, simulated dental condition outcomes, and/or other content in response to one or more prompts. A “prompt” can include inputs and/or portions of inputs with tasks embedded therein. In some embodiments, the 3D model generator 242 can translate interactions (e.g., from a user of a user device displaying the UI generated by UI controller 130) into prompts by framing the interactions into generatively analytical format, e.g., a format that can be input into a large language model. In some embodiments, the 3D model generator 242 can generate and/or provide a prompt to the generative AI model, instructing the generative AI model to generate a 3D model of the patient's dental arch(es) including the indicators of potential future dental condition(s). In some embodiments, the 3D model generator 242 can executed as be part of the scanner (e.g., of oral state capture system 110 of FIG. 1A-B).

Applicant hereby incorporates by reference the following application as if set forth fully here, as an example techniques to improve, update, and/or optimize a 3D dental model, including an ML model to reconstruct a 3D dental from 2D image(s): US. Pat. Pub. No. 20210186659A1, published on Jun. 24, 2021, issued on Aug. 15, 2023, U.S. Pat. No. 11,723,749 B2.

Applicant hereby incorporates by reference the following application as if set forth fully here, as an example of presenting a generated three-dimensional model of a dental arch together with the indications of dental conditions in a GUI: U.S. Pat. Pub. No. 20220202295A1, published on Jun. 30, 2022, issued on Oct. 29, 2024, U.S. Pat. No. 12,127,814 B2.

In some embodiments, the UI controller 130 can enable user interaction with the 3D model. For example, the UI controller 130 can include a scale (e.g., a sliding scale, or a dropdown list) enabling the user to select a level or range of severity. For example, a user could select a range of severity corresponding to indications of predicted tooth wear that exceed 0.5 mm. The UI controller 130 can identify the indication(s) matching the identified scale, and modify the UI to display the indication(s) that match the identified scale. In some embodiments, the UI controller 130 can display colors corresponding to the indications of predicted dental condition progression, where the colors corresponding to the level or range of severity (e.g., indications that predict dental condition progression within a highest severity range can be displayed or highlighted in red, indications that predict dental condition progression within a middle severity range can be displayed or highlighted in yellow, and indications that predict dental condition progression a threshold can be displayed or highlighted in green). In some embodiments, the lowest severity range may not be displayed at all. In some embodiments, the UI controller 130 can include a flag on the center of a tooth to indicate an indication of predicted dental condition progression, and the UI controller 130 can provide the user with more information regarding that indication in response to a user interaction (e.g., the user hovers their mouse over the flag, or the user clicks on the flag). In some embodiments, the UI controller 130 enables a user (e.g., a dental professional) to add, edit, or remove indications of predicted dental condition(s).

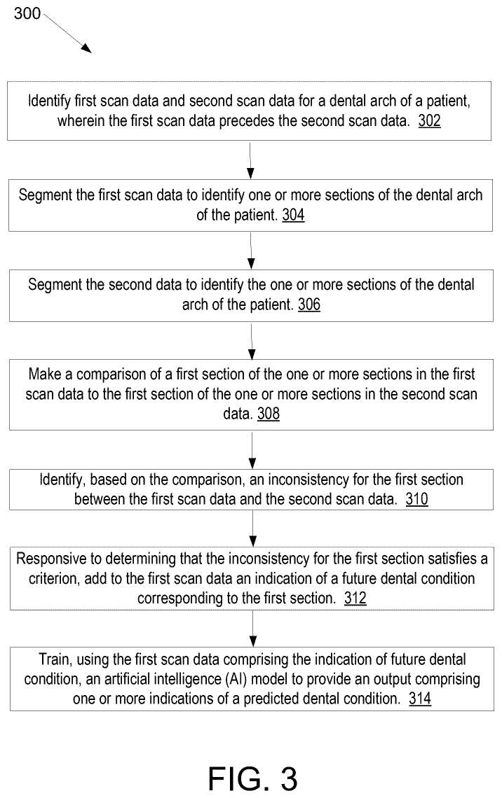

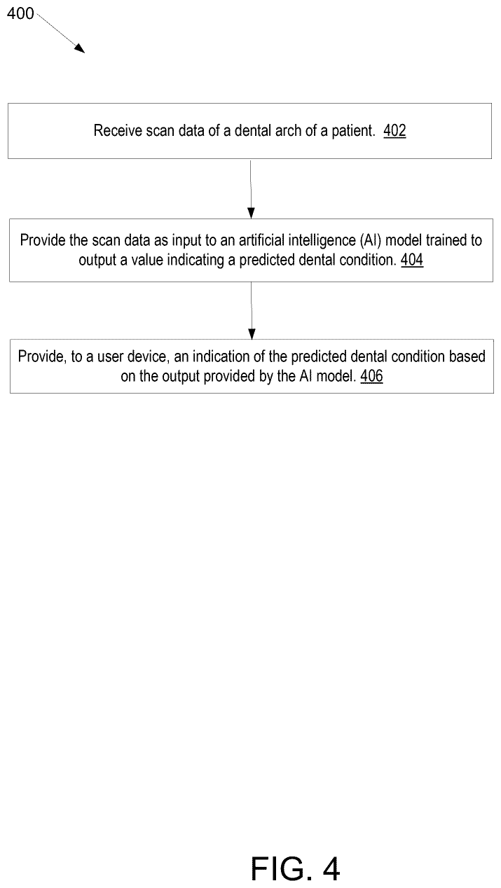

FIGS. 3-4 illustrates flow diagram of example methods 300-400 for generating training data by automatically labeling scan data with indications of future dental condition(s) and/or for providing indications of predicted dental condition(s) in a patient, in accordance with some embodiments of the present disclosure. One or more of methods 300-400 may be performed by a processing device that may include hardware, software, or a combination of both. The processing device may include one or more central processing units (CPUs), graphics processing units (GPUS), field-programmable gate arrays (FPGAs), application-specific integrated circuits (ASICs), or the like, or any combination thereof. In one embodiment, one or more of methods 300-400 may be performed by the processing devices and the associated algorithms, e.g., as described in conjunction with FIG. 1A-B. In embodiments, one or more of methods 300-400 is performed by processing logic comprising hardware, software, firmware, or a combination thereof. In certain embodiments, one or more of methods 300-400 may be performed by a single processing thread.

Alternatively, one or more of methods 300-400 may be performed by two or more processing threads, each thread executing one or more individual functions, routines, subroutines, or operations of the method. In an illustrative example, the processing threads implementing one or more of methods 300-400 may be synchronized (e.g., using semaphores, critical sections, and/or other thread synchronization mechanisms). Alternatively, the processing threads implementing one or more of methods 300-400 may be executed asynchronously with respect to each other. Therefore, while FIGS. 3-4 and the associated descriptions list the operations of methods 300-400 in a certain order, in some embodiments, at least some of the described operations may be performed in parallel and/or in a different order. In some embodiments one or more operations of one or more of methods 300-400 is not performed.

FIG. 3 illustrates flow diagram of an example method 300 for generating training data by automatically labeling scan data with indications of a future dental condition, in accordance with some embodiments of the present disclosure. At block 302, processing logic can identify first scan data and second scan data for a dental arch of a patient. The first scan data and the second scan data can each correspond to a scan performed at distinct points in time. The scan corresponding to the first scan data can be precede the scan corresponding to the second scan data. For example, the first scan data can correspond to a scan (e.g., an intraoral scan) performed at a first point in time (e.g., during a visit to a dentist's office), and the second scan data can correspond to a scan performed at a second point in time (e.g., during a second visit to a dentist's office, a few months after the first visit). In some embodiments, the scan data can correspond to oral state data 151 of FIG. 1A-B.

At block 304, processing logic can segment the first scan data to identify one or more sections of the dental arch of the patient. At block 306, processing logic can segment the second scan data to identify the one or more sections of the dental arch of the patient. Each of the one or more sections can correspond to individual teeth, or to a region in the patient's dental arch corresponding to an individual tooth (e.g., if the tooth is missing, the section corresponds to the region in the dental arch where the missing tooth was once located). In some embodiments, processing logic can segment the first scan and/or the second scan as described with respect to segmentation module 213 of FIG. 2. In some embodiments, the segmentation information can be stored in segmentation data 153 of FIG. 1A-B.

At block 308, processing logic can make a comparison of a first section of the one or more sections in the first scan data to the first section of the one or more sections in the second scan data. That is, based on the segmentation information, processing logic can identify a first occurrence of a section (e.g., an individual tooth) in the first scan data and a second occurrence of the same section (e.g., the same tooth) in the second scan data. Processing logic can compare the scan data corresponding to each occurrence of the section (e.g., tooth). In some embodiments, processing logic can perform the comparison as described with respect to comparison module 226 of FIG. 2.

At block 310, processing logic can identify, based on the comparison, an inconsistency for the first section between the first scan data and the second scan data. In some embodiments, the inconsistency can correspond to tooth wear, gum recession, chipped tooth, bruxism, gastroesophageal reflux disease (GERD), gingival inflammation, a missing attachment, and/or other sources causing changes in the dental arch of the patient over time. In some embodiments, the scan data can be color data, and the inconsistency can correspond to tooth staining, loss of vitality, poor oral care, fluorosis, tooth decay, gingivitis, soft tissue lesion, gingival recession, gingival inflammation, and/or other sources causing changes in the dental arch of the patient over time. In some embodiments, processing logic can identify an inconsistency as described with respect to comparison module 226 of FIG. 2.

At block 312, responsive to determining that the inconsistency for the first section satisfies a criterion, processing logic can add to the first scan data an indication of a future dental condition corresponding to the first section. For example, the indication can be added to scan data (e.g., oral state data 151 of FIG. 1A-B) corresponding to the first scan. The indication can be a label added to the corresponding tooth that exhibited the inconsistency between the first scan and the second scan.

In some embodiments, processing logic can determine that the inconsistency for the first section satisfies the criterion by identifying a cause of the inconsistency, and determining that the cause corresponds to a list of predetermined causes. In some embodiments, to identify a cause of the inconsistency, processing logic can identify patient data corresponding to the patient (e.g., patient data 156 of FIG. 1A-B). The patient data can include indications of events that occurred in the timespan between the two scans corresponding to the first scan data and the second scan data. The events can include, for example, a particular diagnosis related to the patient's dental health, restorative dental work, or another event that is related to the patient's dental health (e.g., an accident that caused tooth damage). Processing logic can identify the event and the corresponding tooth (or teeth) affected by the event(s) to identify a potential cause of the inconsistency for a particular section (e.g., tooth). Processing logic can compare the cause to a list of causes to determine whether the inconsistency satisfies the criterion. In some embodiments, the list of causes can be causes that are related to one or more dental conditions, and processing logic can determine that the cause of the inconsistency satisfies the criterion if the identified cause is included in the list of causes. In some embodiments, the list of causes can be causes that are not related to progressive dental conditions (e.g., restorative work), and processing logic can determine that the cause of the inconsistency satisfies the criterion if the identified cause is not included in the list of causes.

In some embodiments, processing logic can determine that the inconsistency for the first section satisfies the criterion by identifying a size of the inconsistency, and determining that the size exceeds a threshold size. In some embodiments, processing logic can identify the size of the inconsistency based on the delta map (e.g., generated by change identification module 222 of FIG. 2 and/or stored in delta map 158 of FIG. 1A-B). In some embodiments, processing logic can identify the size of the inconsistency by comparing the scan data from the first scan corresponding to a particular tooth to scan data from the second scan corresponding to the particular tooth. In some embodiments, an inconsistency that has a size below the threshold size may not be indicative of a predicted dental condition, and may be due to an error in the imaging process, for example.

At block 314, processing logic can train, using the first scan data comprising the indication of a future dental condition, an AI model to provide an output that includes one or more indications of a predicted dental condition. In some embodiments, processing logic can generate a training dataset that includes the first scan data and a set of inconsistencies including the first inconsistency in the first section. Processing logic can retrain, using the generated training dataset, the AI model to provide the output that includes the one or more indications of a predicted dental condition.

In some embodiments, processing logic generate a delta map that includes inconsistencies corresponding to each of the sections of the dental arch. Processing logic can optionally normalize the delta map to a predetermined time period.

FIG. 4 illustrates a flow diagram of an example method 400 for providing indications of a predicted dental condition in a patient, in accordance with some embodiments of the present disclosure.

At block 402, processing logic receives scan data of a dental arch of a patient. In some embodiments, the scan data can include one or more three-dimensional meshes, one or more two-dimensional scans, and/or one or more occlusal maps. In some embodiments, the scan data can include multiple bite positions of the dental arch of the patient. In some embodiments, the scan data can include information on opposing teeth. For example, a hard material crown in the opposing jaw may cause faster tooth wear. In some embodiments, the scan data of the dental arch of the patient comprises scan data collected from a first scan performed at a first point in time, and does not comprise scan data collected form a second scan performed at a second point in time. That is, the scan data of the dental arch of the patient can correspond only to a scan (or multiple scans) performed at a single point in time (e.g., during a single visit to a dental practitioner). In some embodiments, the scan data can include only current scan data reflecting the current condition of the patient's dental arch.

In some embodiments, processing logic can segment the scan data into individual teeth, as described throughout (e.g., as described with respect to segmentation module 213 of FIG. 2).

At block 404, processing logic provides the scan data as input to an AI model trained to output a value indicating a predicted dental condition. In some embodiments, processing logic provides the segmented scan data as input to the AI model. In some embodiments, the scan data is segmented into individual teeth before being provided to the AI model.

In some embodiments, the AI model is trained using a training dataset that has been automatically labeled with labels indicating a future dental condition based on a comparison of first scan data collected during a first scan performed at a first point in time to second scan data collected during a second scan performed at a second point in time, wherein the comparison indicates the dental condition. An embodiment of the automatic labeling of the training dataset is further described with respect to FIG. 3. In some embodiments, the first scan data and predate the second scan data. In some embodiments, the comparison can identify an inconsistency between the first scan data and the second scan data. In some embodiments, the inconsistency can satisfy a criterion. For example, the inconsistency can satisfy the criterion if a list of predetermined causes includes the cause of the inconsistency. That is, processing logic can identify a cause of the inconsistency, and can determine that the inconsistency satisfies the criterion if the cause is on the list of predetermined causes. As another example, the inconsistency can satisfy the criterion if a size of the inconsistency exceeds a threshold size. In some embodiments, the inconsistency corresponds to at least one of tooth wear, gum recession, chipped tooth, bruxism, gastroesophageal reflux disease (GERD), gingival inflammation, or a missing attachment. In some embodiments, the first scan data and the second scan data can include color data, and the inconsistency can correspond to at least one of tooth staining, loss of vitality, poor oral care, fluorosis, tooth decay, gingivitis, or a soft tissue lesion.

At block 406, processing logic provides, to a user device (e.g., device 160 of FIGS. 1A-B), an indication of a predicted dental condition based on output provided by the AI model. In some embodiments, the indication corresponds to the output from the AI model. In some embodiments, the indication is determined based on processing of the output provided by the AI model using a function. For example, processing logic can receive, from the AI model, the value indicating a predicted dental condition, and can determine, based on the value, the indication of a predicted dental condition. In some embodiments (e.g., in embodiments in which the scan data is segmented into individual teeth prior to being provided to the AI model), the indication of the predicted dental condition can correspond to a particular tooth or gingival portion.

In some embodiments, the indication of the predicted dental condition includes an estimated amount of progression of the predicted dental condition. For example, the estimated amount of progression of the predicted dental condition can be a measurement of tooth wear. In some embodiments, the indication of progression of the predicted dental condition can include an estimated amount of the progression of the predicted dental condition for a particular time period (e.g., for the next 6 months, for the next 12 months, for the next 2 years, etc.).

In some embodiments, the indication of the predicted dental condition corresponds to a first value of a plurality of values provided by the AI model, and the first value corresponds to a highest estimated amount of the predicted dental condition. In some embodiments, the AI model can provide a plurality of values, each one corresponding to an estimated amount of progression of the predicted dental condition. In such embodiments, processing logic can identify an indication of the one or more indications of predicted dental condition for a particular tooth or gingival portion (e.g., as identified during segmentation). The identified indication can be the indication that has the highest amount of progression of the predicted dental condition. Processing logic can provide, to the user device, the identified indication of the predicted dental condition for the particular tooth or gingival region.

In some embodiments, a heat map is provided for display on the user device. The heat map can reflect an estimated amount of progression of the predicted dental condition corresponding to the value indicating the predicted dental condition. In some embodiments, the estimated amount of progression of the predicted dental condition can correspond to an aggregation of a plurality of estimated amounts of progression of predicted dental conditions. The plurality of estimated amounts of progression of the predicted dental condition can correspond to a subset of a plurality of values indicating predicted dental condition provided by the AI model. The subset can correspond to a particular tooth of the dental arch of the patient.

In some embodiments, processing logic can generate a heat map of one or more indications of a predicted dental condition based on one or more values output by the AI model. The heat map can reflect an estimated amount of progression of the predicted dental condition of the one or more values, e.g., for a particular tooth or gingival region. The heat map can represent the predicted dental condition, e.g., using colors to indicate the severity or magnitude (e.g., the amount) of the predicted dental condition. In some embodiments, the heat map can be integrated to a 2D or 3D model of the patient's dental arch, and can add colors corresponding to the indication(s) of predicted dental condition. In some embodiments, processing logic can provide, for display on the user device, the heat map (e.g., as a 2D or 3D model).

In some embodiments, processing logic can aggregate the estimated amount of progression of predicted dental condition of the one or more indications for the particular tooth or gingival region based on one or more values output by the AI model. In some embodiments, the trained AI model can receive input corresponding to the occlusal direction or from multiple directions. The trained AI model can output one or more indications of a predicted dental condition corresponding to each of the multiple directions. Processing logic can aggregate the one or more indications corresponding to each direction for a particular to generate a single value of predicted dental condition for that tooth or gingival region. For example, the trained AI model can output predicted tooth wear for a tooth of 0.5 mm based on input generated from a first direction, and a second predicted tooth wear for the tooth of 1.0 mm based on input generated from a second direction. The directions can correspond to the positioning of the scanning apparatus, for example. The processing logic can aggregate the outputs (e.g., 0.5 mm and 1.0 mm) to generate a singled predicted outcome for that tooth. The processing logic can aggregate the outputs using an average, or maximum, for example.

In some embodiments, at least one of the indications of the one or more indications of a predicted dental condition includes an estimated amount of progression of predicted dental condition for a particular time period. In some embodiments, processing logic can provide, as further input to the AI model, a time frame for the predicted dental condition. In some embodiments, the time frame can be provided by a user of the user device. The output of the AI model can correspond to the time frame.

In some embodiments, the indication of predicted dental condition can include a range of values corresponding to a severity range of the dental condition. In some embodiments, processing logic can receive (e.g., as user input from the user device) a first indication of a severity level. Processing logic can provide, for display on the user device, a first value of the range of the values.

In some embodiments, processing logic provides, to a user device, the one or more indications of a predicted dental condition. In some embodiments, processing logic can provide the one or more indications in a table, e.g., a list of indications. In some embodiments, processing logic can generate or identify a 3D model of the patient's dental arch, and can add the one or more indications to the 3D model (e.g., as described with respect to UI controller 130). In some embodiments, the AI model is a generative AI model that is trained to generate a 3D mesh to display the value indicating the predicted dental condition on a corresponding tooth or gingival region.

In some embodiments, processing logic can identify a training dataset that includes scan data. The scan data can include labels indicating a future dental condition. For example, in some embodiments, the labels can be added to the scan data, e.g., as described with respect to automatic labeling engine 120 of FIGS. 1-2. Processing logic can train, using the training dataset, the AI model to provide the one or more indications of a predicted dental condition.

In some embodiments, processing logic can provide, to the user device, the indication that is identified as corresponding to the highest amount of progression of the predicted dental condition.

In some embodiments, the AI model is a generative AI model that is trained to generate a three-dimensional mesh to display the one or more indications of a predicted dental condition on corresponding identified teeth.

In some embodiments, processing logic receives updated scan data of the dental arch of the patient. The timing of the updated scan data can correspond to the one or more indications of a predicted dental condition provided by the AI model. As an illustrative example, for scan data generated at time T, the AI model can provide one or more indications of predicted tooth wear at time T+6 months. The updated scan data can be generated at time T+6 months. Processing logic can compare the one or more indications of predicted dental condition (e.g., based on scan data generated at time T) to the updated scan data (e.g., generated at time T+6 months). Processing logic can determine, based on the comparison, one or more inconsistencies between the updated scan data and the one or more indications. For example, processing logic can determine that the updated scan data includes more severe tooth wear for a particular tooth than was included in the one or more indications. As another example, processing logic can determine that the updated scan data does not include tooth wear at a particular tooth, contrary to the one or more indications of predicted tooth wear. Processing logic can determine a prognosis factor corresponding to the one or more inconsistencies. The prognosis factor can offset the inconsistencies between the originally predicted dental condition provided by the AI model and the actual dental condition experienced by the patient.

In some embodiments, processing logic can provide, as input to the AI model, the updated scan data. Processing logic can receive, as output from the AI model, one or more updated indications of a predicted dental condition. Processing logic can apply the prognosis factor that corresponds to the inconsistencies, to the one or more updated indications of a predicted dental condition. That is, processing logic can offset the inconsistencies between the predictions output by the AI model and the actual dental condition experienced by the patient. Processing logic can provide, to the user device, the one or more updated indications of the predicted dental condition. In some embodiments, processing logic can provide the updated scan data to AI model, without applying the prognosis factor.

FIG. 5 illustrates workflows for training and using one or more artificial intelligence models to provide indications of a predicted dental condition, in accordance with some embodiments of the present disclosure. The illustrated workflows include a model training workflow 505 and a model application workflow 517. The model training workflow 505 is to train one or more AI models (e.g., deep learning models, generative models, etc.) to provide indications of predicted dental condition(s). The model application workflow 517 is to apply the one or more trained AI models to or provide indications of predicted dental condition(s).

One type of artificial intelligence model that may be used is an artificial neural network, such as a deep neural network. Artificial neural networks generally include a feature representation component with a classifier or regression layers that map features to a desired output space. A convolutional neural network (CNN), for example, hosts multiple layers of convolutional filters. Pooling is performed, and non-linearities may be addressed, at lower layers, on top of which a multi-layer perceptron is commonly appended, mapping top layer features extracted by the convolutional layers to decisions (e.g. classification outputs). Deep learning is a class of artificial intelligence algorithms that use a cascade of multiple layers of nonlinear processing units for feature extraction and transformation. Each successive layer uses the output from the previous layer as input. Deep neural networks may learn in a supervised (e.g., classification) and/or unsupervised (e.g., pattern analysis) manner. Deep neural networks include a hierarchy of layers, where the different layers learn different levels of representations that correspond to different levels of abstraction. In deep learning, each level learns to transform its input data into a slightly more abstract and composite representation. In an image recognition application, for example, the raw input may be a matrix of pixels; the first representational layer may abstract the pixels and encode edges; the second layer may compose and encode arrangements of edges; the third layer may encode higher level shapes (e.g., teeth, gingiva, enamel, etc.); and the fourth layer may recognize that the image contains a face or define a bounding box around teeth in the image. Notably, a deep learning process can learn which features to optimally place in which level on its own. The “deep” in “deep learning” refers to the number of layers through which the data is transformed. More precisely, deep learning systems have a substantial credit assignment path (CAP) depth. The CAP is the chain of transformations from input to output. CAPs describe potentially causal connections between input and output. For a feedforward neural network, the depth of the CAPs may be that of the network and may be the number of hidden layers plus one. For recurrent neural networks, in which a signal may propagate through a layer more than once, the CAP depth is potentially unlimited.

Training of a neural network may be achieved in a supervised learning manner, which involves feeding a training dataset consisting of labeled inputs through the network, observing its outputs, defining an error (by measuring the difference between the outputs and the label values), and using techniques such as deep gradient descent and backpropagation to tune the weights of the network across all its layers and nodes such that the error is minimized. In many applications, repeating this process across the many labeled inputs in the training dataset yields a network that can produce correct output when presented with inputs that are different than the ones present in the training dataset. In high-dimensional settings, such as large images, this generalization is achieved when a sufficiently large and diverse training dataset is made available.

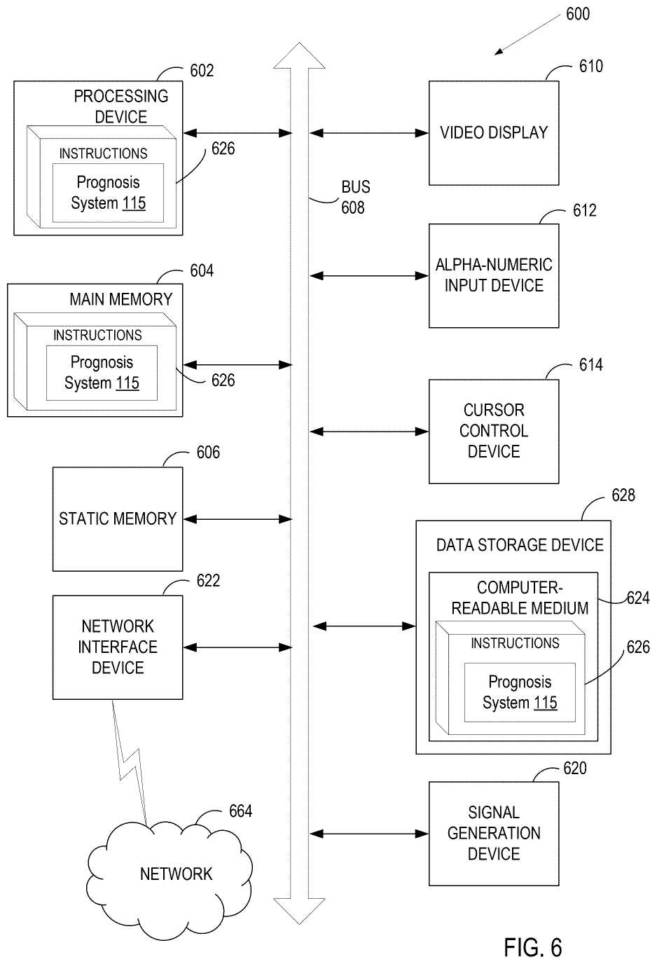

The model training workflow 505 and the model application workflow 517 may be performed by processing logic executed by a processor of a computing device (e.g., computing device 105 of FIG. 1A-B or a separate computing device). These workflows 505, 517 may be implemented, for example, by one or more modules executed on a processing device 602 of computing device 600 shown in FIG. 6.

For the model training workflow 505, training dataset 510 containing hundreds, thousands, tens of thousands, hundreds of thousands, or more images (e.g., scan data and/or additional patient data) may be provided. Training dataset 510 can include oral state data (e.g., scan data) that includes labels indicating areas of a future dental condition. For example, training dataset 510 can include scan data processed by input preprocessing engine 112 of FIGS. 1-2, and/or labeled by automatic labeling engine 120 of FIGS. 1-2. In some embodiments, training dataset 510 can include oral state data (e.g., scan data) of a patient generated at a first point in time, and the delta map indicating the changes in one or more teeth between the first scan and a second scan taken at a second point in time (e.g., a few months after the first scan). In some embodiments, training dataset 510 can include a dataset corresponding to the occlusal map. In some embodiments, multiple dataset from various view directions (e.g., corresponding to the positioning of the scanning apparatus). In some embodiments, training dataset 510 can include additional data with labels, such as mesh segment, height map (e.g., corresponding to the mesh segment), surface normal directions, occlusion data (e.g., occlusal clearance to the other jaw), color data, patient data, and/or other relevant data. In some embodiments, training dataset 510 can include labeled 3D color models generated from intraoral scan data of the dentition of a patient and/or color 2D images. In some embodiments, the training dataset 510 can be stacked as 2D data with multiple channels. The channels can correspond to the relevant data, such as color, surface normal, occlusal distance, NIRI, etc. In some embodiments, some or all of the data may be labeled with segmentation information, e.g., as described with respect to input preprocessing engine 112 of FIGS. 1-2. The segmentation information may identify features, such as individual teeth or a region of the mouth corresponding to individual teeth.

At block 538, data from the training dataset 510 may be used to train one or more artificial intelligence models to provide indications of a predicted future dental condition(s). In some embodiments, the indications can be, or include, indications of progressing clinical findings, such as gingival recession and/or gingival inflammation, for example. The training dataset 510 for providing indications of gingival recession and/or gingival inflammation may not include occlusal map data, as occlusal map data may not be applicable to predicting future gingival recession and/or gingival inflammation. The training dataset containing hundreds, thousands, tens of thousands, hundreds of thousands, or more data points can be used to form the training dataset 510. In embodiments, up to millions of scan data are included in a training dataset. In some embodiments, at block 538, data from training dataset 510 may be used to train one or more artificial intelligence models to generate a 3D model of the patient's dental arch(es) that displays the teeth after the predicted future dental condition(s) has occurred.

Training may be performed by inputting one or more data points into the artificial intelligence model one at a time. The data that is input into the artificial intelligence model may include a single layer or multiple layers. In some embodiments, a recurrent neural network (RNN) is used. In such an embodiment, a second layer may include a previous output of the artificial intelligence model (which resulted from processing a previous input).

The artificial intelligence model processes the input to generate an output. An artificial neural network includes an input layer that consists of values in a data point. The next layer is called a hidden layer, and nodes at the hidden layer each receive one or more of the input values. Each node contains parameters (e.g., weights) to apply to the input values. Each node therefore essentially inputs the input values into a multivariate function (e.g., a non-linear mathematical transformation) to produce an output value. A next layer may be another hidden layer or an output layer. In either case, the nodes at the next layer receive the output values from the nodes at the previous layer, and each node applies weights to those values and then generates its own output value. This may be performed at each layer. A final layer is the output layer, where there is one node for each class, prediction and/or output that the artificial intelligence model can produce. For example, for an artificial neural network being trained to output gingival recession measurement and/or categorization for each tooth.

Processing logic may then compare the generated measurements and/or categorizations to the known condition and/or label that was included in the training data item. Processing logic determines an error based on the differences between the output probability map and/or label(s) and the provided probability map and/or label(s). Processing logic adjusts weights of one or more nodes in the artificial intelligence model based on the error. An error term or delta may be determined for each node in the artificial neural network. Based on this error, the artificial neural network adjusts one or more of its parameters for one or more of its nodes (the weights for one or more inputs of a node). Parameters may be updated in a back propagation manner, such that nodes at a highest layer are updated first, followed by nodes at a next layer, and so on. An artificial neural network contains multiple layers of “neurons,” where each layer receives input values from neurons at a previous layer. The parameters for each neuron include weights associated with the values that are received from each of the neurons at a previous layer. Accordingly, adjusting the parameters may include adjusting the weights assigned to each of the inputs for one or more neurons at one or more layers in the artificial neural network.

Once the model parameters have been optimized, model validation may be performed to determine whether the model has improved and to determine a current accuracy of the model. After one or more rounds of training, processing logic may determine whether a stopping criterion has been met. A stopping criterion may be a target level of accuracy, a target number of processed data items from the training dataset, a target amount of change to parameters over one or more previous data points, a combination thereof and/or other criteria. In one embodiment, the stopping criteria is met when at least a minimum number of data points have been processed and at least a threshold accuracy is achieved. The threshold accuracy may be, for example, 70%, 80% or 90% accuracy. In one embodiment, the stopping criteria is met if accuracy of the artificial intelligence model has stopped improving. If the stopping criterion has not been met, further training is performed. If the stopping criterion has been met, training may be complete. Once the artificial intelligence model is trained, a reserved portion of the training dataset 510 may be used to test the model. Testing the model can include performing unit tests, regression tests, and/or integration tests.

Once one or more trained AI models are generated, they may be stored in model storage 545. Multiple AI models can be trained and used in combination. For example, model training workflow 505 can train an AI model to provide one or more indications of predicted future dental condition(s), and/or a generative AI model to generate a 3D model of the patient's dental arch(es) that illustrates one or more predictions of future dental condition(s). In some embodiments, the generated 3D model can display the teeth of the patient's dental arch(es) after the identified predicted future dental condition(s) has occurred. The first AI model can output value(s) indicating a likelihood and/or severity of predicted dental condition(s) for one or more regions (e.g., teeth) of the patient's dental arch. The generative AI model can output a 3D model of the patient's dental arch that includes the one or more indications of predicted dental condition(s). In some embodiments, at block 538, processing logic can train a single AI model that receives, as input, segmented scan data of representing a region of a patient's dental arch (e.g., representing a single tooth), and can output a value indicating a likelihood and/or a severity of predicted the dental condition(s) for that tooth.

In some embodiments, model application workflow 517 includes one or more trained AI model(s) 570. These logics may be implemented as separate artificial intelligence models or as a single combined artificial intelligence model, in embodiments. However, each of these logics may include distinct higher level layers of the deep neural network that are trained to generate different types of outputs.

In some embodiments, a patient, a dental professional (e.g., a doctor, dentist, hygienist, or technician), and/or another individual may perform a scan of a patient's dental arch(es) at a single point in time (e.g., during a visit to the dentist). The scan can be, for example, an intraoral scan, e.g., as described with respect to FIG. 1A-B. The data generated by the scan can be preprocessed (e.g., by input processing engine 112 of FIGS. 1-2) to segment the scan data. The data generated by the scan (and optionally preprocessed by input preprocessing engine 112) can correspond to oral state data 548, and/or oral state data 151 of FIG. 1A-B.

In some embodiments, the dental professional may have previously captured a scan (e.g., intraoral scan, CBCT scan, etc.), and/or may have other patient data, such as the patient's chart, the patient's previous diagnoses, the patient's previous treatments, the patient's answers to a questionnaire (optionally including a history of patient's answers), 2D image data of the patient's smile showing their teeth (e.g., as captured by a mobile computing device of the patient), and/or the patient's occlusion data, which may correspond to patient data 554. Oral state data 548 and patient data 554 may be combined to form input data 562. Some or all of input data 562 may be processed by a segmenter (e.g., as described with respect to segmentation module 213 of FIG. 2). The segmenter may produce segmentation information, e.g., identifying individual teeth or regions in the dental arch corresponding to individual teeth. The segmentation information can be included in input data 562.

Input data 562 can be provided as input to AI model 570. AI model 570 may produce output 571, which may include a value indicating a likelihood of future dental condition(s). In some embodiments, AI model 570 can include two (or more) AI models, each AI model providing output for scan data corresponding to a different view direction. Output aggregator 776 may aggregate outputs corresponding to each view direction to generate an aggregated output 578. The aggregated output 578 can the predicted dental condition(s) for that particular tooth or region of the dental arch. Thus, the model application workflow 717 may produce, as aggregated output 578, a single value indicating the predicted future dental condition(s) for a particular tooth or region of the dental arch. In some embodiments, AI model 570 can include a generative AI model that generates, as part of output 571, a 3D model of the predicted future state of the patient's dental arch(es). The 3D model can show a visualization of the patient's dental arch(es) after the dental condition(s) has occurred.

In some embodiments, UI controller 130 of FIGS. 1-2 can use the output 571 and/or aggregated output 578 to generate and/or display a UI to provide the dental condition prognosis to a user device.

FIG. 6 illustrates a diagrammatic representation of a machine in the example form of a computing device 600 within which a set of instructions, for causing the machine to perform any one or more of the methodologies discussed herein, may be executed. In alternative embodiments, the machine may be connected (e.g., networked) to other machines in a Local Area Network (LAN), an intranet, an extranet, or the Internet. The machine may operate in the capacity of a server or a client machine in a client-server network environment, or as a peer machine in a peer-to-peer (or distributed) network environment. The machine may be a personal computer (PC), a tablet computer, a set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine. Further, while only a single machine is illustrated, the term “machine” shall also be taken to include any collection of machines (e.g., computers) that individually or jointly execute a set (or multiple sets) of instructions to perform any one or more of the methodologies discussed herein. In one embodiment, the computing device 600 corresponds to any computing device of FIG. 1A-B.

The example computing device 600 includes a processing device 602 (e.g., a CPU), a main memory 604 (e.g., read-only memory (ROM), flash memory, dynamic random access memory (DRAM) such as synchronous DRAM (SDRAM), etc.), a static memory 606 (e.g., flash memory, static random access memory (SRAM), etc.), and a secondary memory (e.g., a data storage device 628), which communicate with each other via a bus 608.

Processing device 602 represents one or more general-purpose processors such as a microprocessor, central processing unit, or the like. More particularly, the processing device 602 may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processing device 602 may also be one or more special-purpose processing devices such as an application specific integrated circuit (ASIC), a field programmable gate array (FPGA), a digital signal processor (DSP), network processor, or the like. In accordance with one or more aspects of the present disclosure, processing device 602 is configured to execute the processing logic (instructions 626, which may implement the prognosis system 115 of FIG. 1A-B) for performing operations and steps discussed herein. While only a single example processing device is illustrated, the term “processing device” shall also be taken to include any collection of processing devices (e.g., computers) that individually or jointly execute a set (or multiple sets) of instructions to perform any one or more of the methods discussed herein.

The computing device 600 may further include a network interface device 622 for communicating with a network 664. The computing device 600 also may include a video display unit 610 (e.g., a liquid crystal display (LCD) or a cathode ray tube (CRT), an alphanumeric input device 612 (e.g., a keyboard), a cursor control device 614 (e.g., a mouse, a touch-screen control device), and a signal generation device 620 (e.g., a speaker).

The data storage device 628 may include a machine-readable storage medium (or more specifically a non-transitory computer-readable storage medium) 624 on which is stored one or more sets of instructions 626 embodying any one or more of the methodologies or functions described herein. A non-transitory storage medium refers to a storage medium other than a carrier wave. The instructions 626 may also reside, completely or at least partially, within the main memory 604 and/or within the processing device 602 during execution thereof by the computer device 600, the main memory 604 and the processing device 602 also constituting computer-readable storage media.

The computer-readable storage medium 624 may also be used to store a prognosis system 115, which may correspond to the similarly named component of FIG. 1A-B. The computer readable storage medium 624 may also store a software library containing methods for a prognosis system 115. While the computer-readable storage medium 624 is shown in an example embodiment to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any non-transitory medium (e.g., a medium other than a carrier wave) that is capable of storing or encoding a set of instructions for execution by the machine and that cause the machine to perform any one or more of the methodologies of the present disclosure. The term “computer-readable storage medium” shall accordingly be taken to include, but not be limited to, solid-state memories, and optical and magnetic media.

Any of the methods (including user interfaces) described herein may be implemented as software, hardware or firmware, and may be described as a non-transitory machine-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, computer models (e.g., for additive manufacturing) and instructions related to forming a dental device may be stored on a non-transitory machine-readable storage medium.

It should be understood that the above description is intended to be illustrative, and not restrictive. Many other embodiment examples will be apparent to those of skill in the art upon reading and understanding the above description. Although the present disclosure describes specific examples, it will be recognized that the systems and methods of the present disclosure are not limited to the examples described herein, but may be practiced with modifications within the scope of the appended claims. Accordingly, the specification and drawings are to be regarded in an illustrative sense rather than a restrictive sense. The scope of the present disclosure should, therefore, be determined with reference to the appended claims, along with the full scope of equivalents to which such claims are entitled.

The embodiments of methods, hardware, software, firmware, or code set forth above may be implemented via instructions or code stored on a machine-accessible, machine readable, computer accessible, or computer readable medium which are executable by a processing element. “Memory” includes any mechanism that provides (i.e., stores and/or transmits) information in a form readable by a machine, such as a computer or electronic system. For example, “memory” includes random-access memory (RAM), such as static RAM (SRAM) or dynamic RAM (DRAM); ROM; magnetic or optical storage medium; flash memory devices; electrical storage devices; optical storage devices; acoustical storage devices, and any type of tangible machine-readable medium suitable for storing or transmitting electronic instructions or information in a form readable by a machine (e.g., a computer).

Reference throughout this specification to “one embodiment” or “an embodiment” means that a particular feature, structure, or characteristic described in connection with the embodiment is included in at least one embodiment of the disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In the foregoing specification, a detailed description has been given with reference to specific exemplary embodiments. It will, however, be evident that various modifications and changes may be made thereto without departing from the broader spirit and scope of the disclosure as set forth in the appended claims. The specification and drawings are, accordingly, to be regarded in an illustrative sense rather than a restrictive sense. Furthermore, the foregoing use of embodiment, embodiment, and/or other exemplarily language does not necessarily refer to the same embodiment or the same example, but may refer to different and distinct embodiments, as well as potentially the same embodiment.

The words “example” or “exemplary” are used herein to mean serving as an example, instance, or illustration. Any aspect or design described herein as “example’ or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or.” That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Moreover, use of the term “an embodiment” or “one embodiment” or “an embodiment” or “one embodiment” throughout is not intended to mean the same embodiment or embodiment unless described as such. Also, the terms “first,” “second,” “third,” “fourth,” etc. as used herein are meant as labels to distinguish among different elements and may not necessarily have an ordinal meaning according to their numerical designation.

A digital computer program, which may also be referred to or described as a program, software, a software application, a module, a software module, a script, or code, can be written in any form of programming language, including compiled or interpreted languages, or declarative or procedural languages, and it can be deployed in any form, including as a stand-alone program or as a module, component, subroutine, or other unit suitable for use in a digital computing environment. The essential elements of a digital computer a central processing unit for performing or executing instructions and one or more memory devices for storing instructions and digital data. The central processing unit and the memory can be supplemented by, or incorporated in, special purpose logic circuitry or quantum simulators. Generally, a digital computer will also include, or be operatively coupled to receive digital data from or transfer digital data to, or both, one or more mass storage devices for storing digital data, e.g., magnetic, magneto-optical disks, optical disks, or systems suitable for storing information. However, a digital computer need not have such devices.

Digital computer-readable media suitable for storing digital computer program instructions and digital data include all forms of non-volatile digital memory, media, and memory devices, including by way of example semiconductor memory devices, e.g., EPROM, EEPROM, and flash memory devices; magnetic disks, e.g., internal hard disks or removable disks; magneto-optical disks; CD-ROM and DVD-ROM disks.

Control of the various systems described in this specification, or portions of them, can be implemented in a digital computer program product that includes instructions that are stored on one or more non-transitory machine-readable storage media, and that are executable on one or more digital processing devices. The systems described in this specification, or portions of them, can each be implemented as an apparatus, method, or system that may include one or more digital processing devices and memory to store executable instructions to perform the operations described in this specification.

While this specification contains many specific embodiment details, these should not be construed as limitations on the scope of what may be claimed, but rather as descriptions of features that may be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A system comprising:
an intraoral scanner configured to generate scan data of a dental arch of a patient; and
a computing device operatively coupled to the intraoral scanner, the computing device configured to:
receive the scan data;
provide the scan data as input to an artificial intelligence (AI) model trained to output a value indicating a predicted dental condition; and
provide, to a user device, an indication of the predicted dental condition based on the output provided by the AI model.

2. The system of claim 1, wherein the predicted dental condition comprises at least one of: tooth wear, gum recession, gingival inflammation, chipped tooth, bruxism-related damage, gastroesophageal reflux disease (GERD) effects, or a missing attachment.

3. The system of claim 1, wherein the scan data comprises color data, and wherein the predicted dental condition comprises at least one of tooth staining, loss of vitality, poor oral care indicator, fluorosis, tooth decay, gingivitis, or a soft tissue lesion.

4. (canceled)

5. (canceled)

6. The system of claim 1, wherein the computing device is further configured to:
receive, from the AI model, the value indicating the predicted dental condition; and
determine, based on the value, the indication of the predicted dental condition.

7. The system of claim 1, wherein the scan data of the dental arch of the patient comprises scan data collected from a first scan performed at a first point in time, and does not comprise scan data collected from a second scan performed at a second point in time.

8. The system of claim 1, wherein the AI model is trained using a training dataset that has been automatically labeled with labels indicating a future dental condition based on a comparison of first scan data collected during a first scan performed at a first point in time to second scan data collected during a second scan performed at a second point in time, wherein the comparison indicates progression of a dental condition.

9. The system of claim 8, wherein the comparison is used to identify an inconsistency between the first scan data and the second scan data that satisfies a criterion, wherein the inconsistency corresponds to at least one of tooth wear, gum recession, chipped tooth, bruxism, gastroesophageal reflux disease (GERD), gingival inflammation, or a missing attachment.

10. The system of claim 9, wherein the inconsistency satisfies the criterion if at least one of a list of predetermined causes comprises a cause of the inconsistency or a size of the inconsistency exceeds a threshold size.

11. (canceled)

12. (canceled)

13. The system of claim 9, wherein the first scan data and the second scan data comprise color data, and wherein the inconsistency corresponds to at least one of: tooth staining, loss of vitality, poor oral care indicator, fluorosis, tooth decay, gingivitis, or a soft tissue lesion.

14. The system of claim 1, wherein the indication of the predicted dental condition comprises an estimated amount of a progress of the predicted dental condition for a particular time period.

15. (canceled)

16. The system of claim 1, wherein the scan data is segmented into at least one of individual teeth or gingival regions before being provided to the AI model, and wherein the indication of the predicted dental condition corresponds to at least one of a particular tooth or a particular gingival region.

17. The system of claim 1, wherein the indication provided to the user device corresponds to a first value of a plurality of values provided by the AI model, wherein the first value corresponds to a highest estimated amount of a progress of the predicted dental condition.

18. The system of claim 1, wherein a heat map is provided for display on the user device, wherein the heat map reflects an estimated amount of a progress of the predicted dental condition corresponding to the value indicating the predicted dental condition.

19. The system of claim 18, wherein the estimated amount of the progress of the predicted dental condition corresponds to an aggregation of a plurality of estimated amounts of progress of the predicted dental condition, wherein the plurality of estimated amounts of progress of the predicted dental condition correspond to at least a subset of a plurality of values indicating predicted dental condition provided by the AI model, and wherein the subset corresponds to a particular tooth of the dental arch of the patient.

20. The system of claim 1, wherein the AI model is a generative AI model that is trained to generate a three-dimensional mesh to display the value indicating the predicted dental condition on at least one of a corresponding tooth or a corresponding gingival region.

21. (canceled)

22. The system of claim 1, wherein the scan data comprises at least one of data from multiple bite positions of the dental arch of the patient or information on opposing teeth.

23. (canceled)

24. The system of claim 1, wherein the computing device is further configured to:
provide, as further input to the AI model, a time frame for the predicted dental condition, wherein the output from the AI model corresponds to the time frame.

25. (canceled)

26. (canceled)

27. The system of claim 1, wherein the indication of the predicted dental condition comprises a range of values corresponding to a severity range of the predicted dental condition, and wherein the computing device is further configured to:
provide, for display on the user device, a first value of the range of values, wherein the first value corresponds to the an indication of the severity range, wherein the indication is received from the user device, and wherein the first value of the range of values reflects the severity range of the predicted dental condition.

28. The system of claim 1, wherein a prognosis factor corresponds to an inconsistency between updated scan data of the dental arch and the indication of the predicted dental condition, wherein the inconsistency is based on a comparison of the updated scan data of the dental arch to the indication of the predicted dental condition, and wherein a timing of the updated scan data corresponds to the indication of the predicted dental condition based on output provided by the AI model.

29. The system of claim 28, wherein the computing device is further configured to:
provide, as further input to the AI model, the updated scan data; and
provide, to the user device, an updated indication of the predicted dental condition based on updated output provided by the AI model modified by the prognosis factor corresponding to the inconsistency between the updated scan data and the indication of the predicted dental condition.

30. (canceled)

31. A method comprising:
receiving scan data of a dental arch of a patient;
providing the scan data as input to an artificial intelligence (AI) model trained to output a value indicating a predicted dental condition; and
providing, to a user device, an indication of the predicted dental condition based on the output provided by the AI model.

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. A non-transitory computer-readable storage medium comprising instructions that, when executed by a processing device, cause the processing device to:
receive scan data of a dental arch of a patient;
provide the scan data as input to an artificial intelligence (AI) model trained to output a value indicating a predicted dental condition; and
provide, to a user device, an indication of the predicted dental condition based on the output provided by the AI model.

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. (canceled)

105. (canceled)

106. (canceled)

107. (canceled)

108. (canceled)

109. (canceled)

110. (canceled)

111. (canceled)

112. (canceled)

113. (canceled)

114. (canceled)

115. (canceled)

116. (canceled)

117. (canceled)

118. (canceled)

119. (canceled)

120. (canceled)

121. (canceled)

122. (canceled)

123. (canceled)

124. (canceled)

125. (canceled)

126. (canceled)

127. (canceled)

128. (canceled)

129. (canceled)

130. (canceled)

131. (canceled)

132. (canceled)

133. (canceled)

134. (canceled)

135. (canceled)

136. (canceled)

137. (canceled)

138. (canceled)

139. (canceled)

140. (canceled)

141. (canceled)

142. (canceled)

143. (canceled)

144. (canceled)

145. (canceled)

146. (canceled)

147. (canceled)

148. (canceled)

149. (canceled)

150. (canceled)

151. (canceled)

152. (canceled)

153. (canceled)

154. (canceled)

155. (canceled)

156. (canceled)

157. (canceled)

158. (canceled)

159. (canceled)

160. (canceled)

161. (canceled)

162. (canceled)

163. (canceled)

164. (canceled)

165. (canceled)

166. (canceled)

167. (canceled)

168. (canceled)

169. (canceled)

170. (canceled)

171. (canceled)

172. (canceled)

173. (canceled)

174. (canceled)

175. (canceled)
